(19) United States
(12) Patent Application Publication
(10) Pub. No.: US 2015/0080333 A1
(43) Pub. Date: Mar. 19, 2015

(54) HYALURONIC ACID PARTICLES AND THEIR USE IN BIOMEDICAL APPLICATIONS

(71) Applicant: UNIVERSITY OF KANSAS, Lawrence, KS (US)

(72) Inventors: Cory Berkland, Lawrence, KS (US); Amir Fakhari, Overland Park, KS (US)

(21) Appl. No.: 14/334,693

(22) Filed: Jul. 18, 2014

Related U.S. Application Data
(63) Continuation of application No. PCT/US13/22250, filed on Jan. 18, 2013.
(60) Provisional application No. 61/588,002, filed on Jan. 18, 2012.

(51) Int. Cl. A61K 31/728; A61K 9/06; C08B 37/08

(52) U.S. Cl. CPC A61K 31/728; C08B 37/0072; A61K 9/06
USPC 514/54; 536/53

(57) ABSTRACT

Hyaluronic acid particles and their use in biomedical applications are provided. In one embodiment, an HA particle comprising a plurality of free polymer chains extending from a surface of the particle such that the polymer chains are capable of association with polymers or with polymer chains on a surface of other particles is provided.

### CROSS-REFERENCE TO RELATED APPLICATIONS

This application is a continuation of PCT/US2013/22250 filed Jan. 18, 2013 and claims the benefit of U.S. Provisional Patent Application Ser. No. 61/588,002 filed Jan. 18, 2012, which are incorporated herein by reference.

### BACKGROUND

Hyaluronic acid (HA) is one of the major elements in extracellular matrix (ECM) of vertebrate tissues. It is found in almost all body fluids and tissues, such as the synovial fluid, the vitreous humor of the eye, and hyaline cartilage. This biopolymer works as a scaffold, binding other matrix molecules including aggrecan. It is also involved in several important biological functions, such as regulation of cell adhesion and cell motility, manipulation of cell differentiation and proliferation, and providing biomechanical properties of tissues. Several cell surface receptors such as CD44, RHAMM, and ICAM-1 interact with HA influencing cellular processes including morphogenesis, wound repair, inflammation, and metastasis. Moreover, HA is responsible for supporting the viscoelasticity of biofluids (synovial fluid and vitreous humor of the eye) and controlling tissue hydration and water transport. In addition, HA has been found during embryonic development, suggesting materials composed of HA may persuade favorable conditions for tissue regeneration and growth.

HA's characteristics including its consistency, biocompatibility, and hydrophilicity have made it an excellent moisturizer in cosmetic dermatology and skin-care products. Moreover, its unique viscoelasticity and limited immunogenicity have led it to be used in several biomedical applications such as viscosupplementation in osteoarthritis treatment, as a surgical aid in ophthalmology, and for surgical wound regeneration in dermatology. In addition, HA has currently been explored as a drug delivery agent for different routes such as nasal, pulmonary, ophthalmic, topical and parenteral.

Hyaluronic acid performs several structural tasks in the extracellular matrix (ECM) as it binds with cells and other biological components through specific and non-specific interactions. Several extracellular matrix proteins are stabilized upon binding to HA. Specific molecules and receptors that interact with HA are involved in cellular signal transduction; molecules such as aggrecan, versican, and neurocan, and receptors including CD44, RHAMM, TSG6, GHAP, and LYVE-1 are examples of cell components that bind to HA. Between these receptors, CD44 and RHAMM seem to have received more attention since they are found to be involved in cancer metastases. CD44 is a structurally variable and multifunctional cell surface glycoprotein on most cell types and is perhaps the best characterized transmembrane HA receptor so far. Due to its wide distribution and based on current knowledge, CD44 is considered to be the primary HA receptor on most cell types.

Hyaluronic acid also stimulates gene expression in macrophages, endothelial cells, eosinophils, and certain epithelial cells. High molecular weight HA does not seem to be involved in gene expression and only low/intermediate molecular weight HA (2×10⁴-4.5×10⁵ Da) is known to promote gene expression. As an example, HA is also known to have an important role in wound healing and scar formation. Products of HA degradation (low molecular weight HA) are identified to contribute in the scar formation process. Moreover, scar formation was minimized when high molecular weight HA was found in wound fluid during fetal wound healing. These results suggested that the molecular weight of HA plays a significant role in wound healing and scar formation. The findings also suggested that high molecular weight HA favored cell quiescence and supported tissue integrity, whereas production of HA fragments signaled injury and initiated the inflammatory response. Synovial fluid is one of the body fluids containing high molecular weight HA. Lubrication and viscoelasticity are properties of high molecular weight HA in synovial fluid. In a healthy joint, high levels (2-4 g/L) of HA with high molecular weight (approximately 6-7 MDa) are required for synovial fluid to function properly.

Polymer nanotechnology has emerged as an important tool to successfully develop and modify biomaterials for drug delivery and tissue engineering. Methods have been developed to modify surface features and core components of nanoparticles for many applications. For instance, conjugation and optimization of targeting moieties to the surface of nanoparticles has been introduced to enhance targeted delivery of therapeutic agents to cells. Moreover, research studies have been developed to modify the surface of nanoparticles and fabricate biocompatible scaffold constructs for tissue engineering applications. Due to discrete properties of polymers and varied application of nanoparticles, multiple fabrication methods have been developed. However, continuous development of novel nanoparticle fabrication methods has always been required.

### SUMMARY

The present disclosure generally relates to polymers. More particularly, the present disclosure relates to hyaluronic acid particles and their applications.

In one embodiment, an HA particle comprising a plurality of polymer chains extending from a surface of the particle such that the polymer chains are capable of association with polymers or with polymer chains on a surface of other particles is provided.

In another embodiment, a method of forming a colloidal gel without using a chemical reaction comprising: providing a plurality of HA particles, wherein at least one of the HA particles comprises a plurality of polymer chains extending from a surface of the particle such that the polymer chains are capable of association with polymers or with polymer chains on a surface of other particles; adding water to the plurality of HA particles; and allowing the HA particles to associate to form a colloidal gel is provided.

In another embodiment, a colloidal suspension comprising a plurality of HA particles, wherein at least one of the HA particles comprises a plurality of polymer chains extending from a surface of the particle such that the polymer chains are capable of association with polymers or with polymer chains on a surface of other particles is provided.

In another embodiment, a method of modifying viscosity of a polymer solution comprising: providing a polymer solution; and adding a plurality of HA particles to the polymer solution to reach a desired concentration of HA in the polymer solution is provided.

In another embodiment, a dermal filler comprising at least one HA particle is provided.

In another embodiment, a tissue engineering scaffold comprising a colloidal gel, wherein the gel comprises a plurality of HA particles, wherein at least one of the HA particles comprises a plurality of polymer chains extending from a surface of the particle such that the polymer chains are capable of association with polymers or with polymer chains on a surface of other particles is provided.

In another embodiment, a method of increasing a concentration of HA in an HA polymer solution without a corresponding increase in viscosity of the polymer solution comprising: providing an HA polymer solution; and adding a plurality of HA particles to the polymer solution to reach a desired concentration of HA in the polymer solution is provided.

The features and advantages of the present invention will be apparent to those skilled in the art. While numerous changes may be made by those skilled in the art, such changes are within the spirit of the invention.

### DRAWINGS

Some specific example embodiments of the disclosure may be understood by referring, in part, to the following description and the accompanying drawings.

FIG. 1 shows the chemical structure of two glycosaminoglycans (GAGs) made of disaccharide repeats of N-acetylglucosamine and glucuronic acid. (A) Hyaluronic acid (HA). (B) Chondroitin sulfate (CS).

FIG. 2 shows (A) Particle fabrication steps. (B) Before dialyzing, light blue color of the solution indicates formation of nanoparticles.

FIG. 3 shows application of carbodiimide chemistry for HA (or CS) nanoparticle fabrication. EDC activates carboxyl groups available on HA and provides reactive intermediates which react with two primary amines of adipic acid dihydrazide forming peptide bonds and resulting in the neighboring HA chains being chemically crosslinked.

FIG. 4 shows particle formation and the effect of HA Mw on nanoparticles.

FIG. 5 shows Cryo-transmission electron micrographs of hyaluronic acid nanoparticles. Left: nanoparticles made from 17 kDa HA, right: nanoparticles made from 1500 kDa HA.

FIG. 6 shows (A) FTIR spectra of 17 kDa HA polymer (HA-PO) and nanoparticles made from 17 kDa HA (HA-NP). (B) FTIR spectra of CS polymer (CS-PO) and CS nanoparticles (CS-NP).

FIG. 7 shows the use of the TNBS assay as a probe to identify the availability of unreacted primary amine groups on crosslinker (PO=Polymer, NP=Nanoparticles, and XL=crosslinker). Data represent the mean±SD (n=3).

FIG. 8 shows (A) 17 kDa HA polymer solutions (HA-PO) at different concentrations (5%, 15%, 30%, and 45% w/v). (B) Colloidal gels (HA-NP) formed at different HA (17 kDa) nanoparticles concentrations (15%, 30%, and 45% w/v). (C) Carbodiimide chemistry also did not form stable gel at different HA (17 kDa) concentrations. (D) 1500 kDa polymer solutions (HA-PO) at different concentrations (1.4%, 5%, 10, 15% w/v). (E) Nanoparticle suspensions (HA-NP) formed at different HA (1500 kDa) nanoparticles concentrations (1.4%, 5%, 10, 15% w/v). (F) Formation of paste-like materials was observed at 30% and 45% w/v HA (1500 kDa) nanoparticle concentrations.

FIG. 9 shows (Top) Physical entanglement due to the presence of dangling chains on the surface of 17 kDa HA nanoparticles may facilitate formation of stable colloidal gel networks. (Bottom) Due to the absence of dangling chains on 1500 kDa HA nanoparticles, physical entanglement and formation of stable colloidal gel networks did not occur.

FIG. 10 shows that colloidal gel networks did not form when using chondroitin sulfate nanoparticles even at 60% w/v concentration.

FIG. 11 shows the FTIR spectra of HA polymer (HA-PO), HA nanoparticles (HA-NP), and HA colloidal gel (HA-Gel) at 30% w/v concentration. The results indicated that chemical side reactions did not occur during colloidal gel formation (17 kDa HA).

FIG. 12 shows swelling experiment on colloidal gels after 6 hours incubation. Samples with lower nanoparticle concentrations tended to swell more in both water and 0.1 M PBS. Samples tended to swell more in water at all concentrations. Data represent the mean±SD (n=3). * indicates p<0.05.

FIG. 13 shows swelling experiment on colloidal gels after 24 hours incubation. Samples with lower nanoparticle concentrations tended to swell more in both water and 0.1 M PBS. Samples tended to swell more in water at all concentrations. Swelling ratios were higher after 24 hours incubation than swelling ratios after 6 hours incubation. Data represent the mean±SD (n=3). * indicates p<0.05.

FIG. 14 shows samples after fabrication, swollen in deionized water, and in 0.1 M PBS.

FIG. 15 shows compression testing up to 80% stain on colloidal gels after fabrication. A failure point was not observed.

FIG. 16 shows colloidal gels before and after compression testing (80% strain, 0.005 mm/s). After removing the force, samples could recover their initial shape over time (˜5 minutes).

FIG. 17 shows uniaxial compression testing of colloidal gels after swelling in deionized water.

FIG. 18 shows uniaxial compression testing of colloidal gels after swelling in 0.1 M PBS.

FIG. 19 shows calculated Young's modulus for colloidal gels at different conditions (after fabrication, swollen in deionized water, or swollen in 0.1 M PBS). Increasing nanoparticle concentration increased Young's modulus. Samples swollen in water had the highest modulus. Data represent the mean±SD (n=3). * and ** p<0.05 as compared to HA-NP-15% w/v.

FIG. 20 shows calculated shear modulus for colloidal gels at different conditions (after fabrication, swollen in deionized water, or swollen in 0.1 M PBS). Increasing nanoparticle concentration increased shear modulus. Data represent the mean±SD (n=3). * and ** p<0.05 as compared to HA-NP-15% w/v.

FIG. 21 shows calculated E/G for colloidal gel samples at different nanoparticle concentrations (after fabrication, swollen in deionized water, or swollen in 0.1 M PBS). Data represent the mean±SD (n=3). * and ** p<0.05 as compared to HA-NP-15% w/v.

FIG. 22 shows compression testing up to 70% stain on colloidal gels after fabrication in phosphate buffer solutions at different pH values (6.0, 7.4, and 8) and constant ionic strength (150 mM). A failure point was not observed for the samples.

FIG. 23 shows calculated Young's modulus, shear modulus, and E/G for colloidal gels at different pH values and constant ionic strength (150 mM). Increasing pH decreased Young's modulus and shear modulus. Data represent the mean±SD (n=3).

FIG. 24 shows compression testing up to 70% stain on colloidal gels in phosphate buffer solutions at different ionic strength values (100, 150, and 200 mM) and a pH of 7.4. A failure point was not observed for the samples.

FIG. 25 shows calculated Young's modulus, shear modulus, and E/G for colloidal gels at different ionic strength values and constant pH of 7.4. Data represent the mean±SD (n=3). * p<0.05 comparing to HA-NP-15% w/v-pH 7.4-100 mM.

FIG. 26 shows viscoelasticity of colloidal gels: storage modulus over frequency range. Increasing nanoparticle concentration increased storage modulus. Data represent the mean±SD (n=3).

FIG. 27 shows viscoelasticity of colloidal gels: loss modulus over frequency range. Increasing nanoparticle concentration increased loss modulus. Data represent the mean±SD (n=3).

FIG. 28 shows viscoelasticity of colloidal gels: complex modulus over frequency range. Increasing nanoparticle concentration increased complex modulus. Data represent the mean±SD (n=3).

FIG. 29 shows viscoelasticity of colloidal gels: tan delta over frequency range. Data represent the mean±SD (n=3).

FIG. 30 shows rheological evaluation of HA-NP (17 kDa) for nanoparticles in deionized water. Increasing nanoparticle concentration increased shear stress.

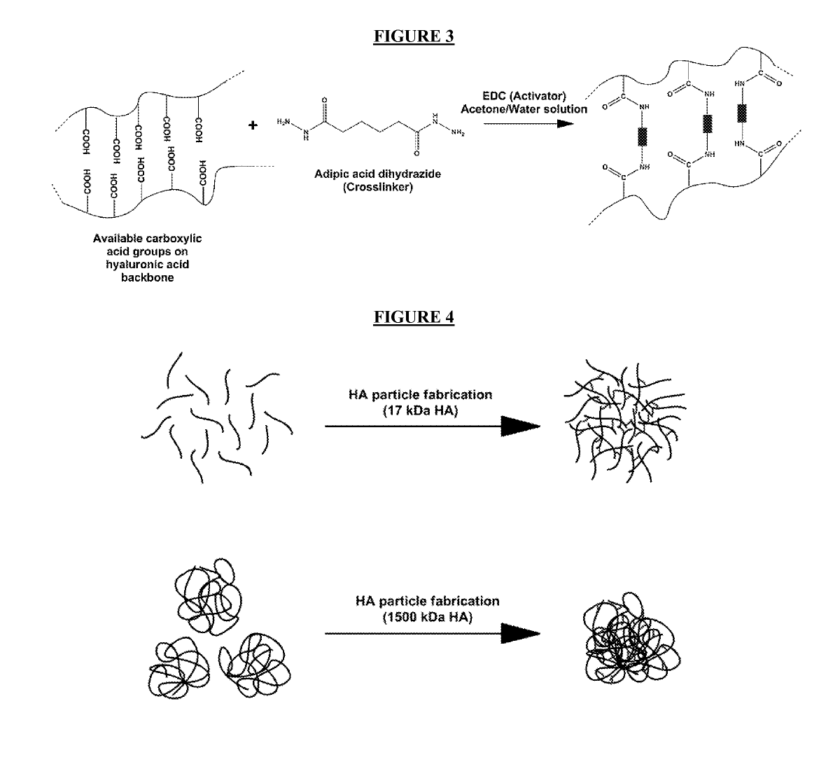

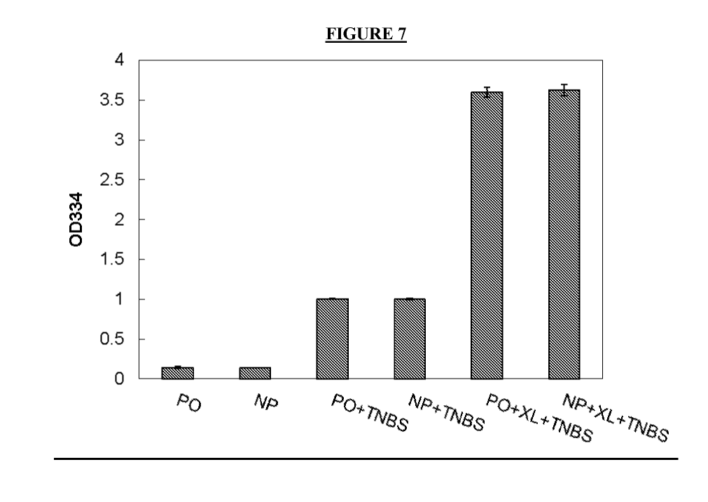

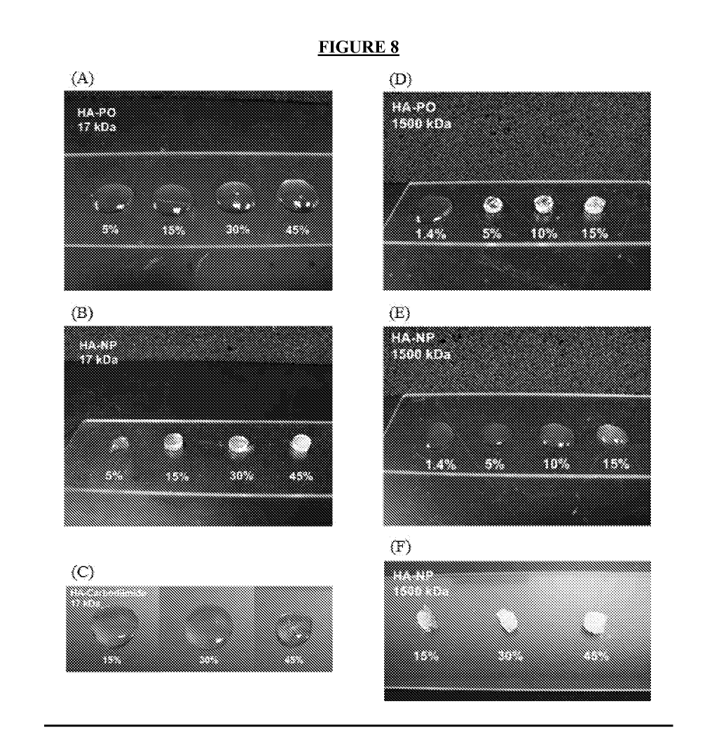

FIG. 3-24 shows rheological evaluation of HA-NP (17 kDa) for nanoparticles in deionized water. Increasing nanoparticle concentration increased viscosity.

FIG. 32 shows the recovery of colloidal gels (15%, 30%, and 45% w/v) after fabrication (A, B, and C) and swollen in 0.1 M PBS (D, E, and F) was determined by applying two compression\decompression cycles with 5 minutes delay time between cycles. Data represent the mean±SD (n=3): C1: 30% strain, 0.005 mm/s. C2: 30% strain, 0.001 mm/s. Five preconditioning cycles were also applied before the recovery experiment at 6% strain, 0.005 mm/s.

FIG. 33 shows colloidal gel recoverability based on rheology. After applying shear stress to the sample (15% w/v nanoparticle concentration), shear recovery was observed after a one minute delay between cycles.

FIG. 34 shows colloidal gel recoverability based on rheology. After applying shear stress to the sample (15% w/v nanoparticle concentration), shear recovery was observed after a one minute delay between cycles.

FIG. 35 shows physical recoverability tests suggested that the colloidal gel networks could be reformed from destroyed colloidal gels.

FIG. 36 shows (Left): HA polymer solution (HA-PO 1500 kDa) at 1.4% w/v concentration. A viscous polymer solution was observed. (Right): HA nanoparticle suspension (HA-NP 1500 kDa) at 1.4% w/v concentration. Viscosity of the nanoparticle suspension was much lower than the polymer solution.

FIG. 37 shows rheological evaluation of HA polymer (1500 kDa) and HA nanoparticle (17 kDa) mixtures. Increasing nanoparticle content in the formulation decreased shear stress.

FIG. 38 shows rheological evaluation of HA polymer (1500 kDa) and HA nanoparticle (17 kDa) mixtures. Increasing nanoparticle content in the formulation decreased viscosity.

FIG. 39 shows rheological evaluation of HA polymer (1500 kDa) and HA nanoparticle (1500 kDa) mixtures. Increasing nanoparticle content in the formulation decreased shear stress.

FIG. 40 shows rheological evaluation of HA polymer (1500 kDa) and HA nanoparticle (1500 kDa) mixtures. Increasing nanoparticle content in the formulation decreased viscosity.

FIG. 41 shows rheological evaluation of HA polymer/nanoparticle mixtures. Viscosity of samples made with 17 kDa HA nanoparticles was greater than the viscosity of the samples made from 1500 kDa HA nanoparticles at all polymer: nanoparticle ratios.

FIG. 42 shows rheological evaluation of HA nanoparticle (1500 kDa) suspensions at different nanoparticle concentrations. Increasing nanoparticle concentration increased shear stress.

FIG. 43 shows rheological evaluation of HA nanoparticle (1500 kDa) suspensions at different nanoparticle concentrations. Increasing nanoparticle concentration increased viscosity.

FIG. 44 shows viscoelasticity of HA polymer (1500 kDa) and HA nanoparticle (17 kDa) mixtures: storage modulus over frequency range. Increasing nanoparticle concentration decreased storage modulus.

FIG. 45 shows viscoelasticity of HA polymer (1500 kDa) and HA nanoparticle (17 kDa) mixtures: loss modulus over frequency range. Increasing nanoparticle concentration decreased loss modulus.

FIG. 46 shows viscoelasticity of HA polymer (1500 kDa) and HA nanoparticle (17 kDa) mixtures: complex modulus over frequency range. Increasing nanoparticle concentration decreased complex modulus.

FIG. 47 shows viscoelasticity of HA polymer (1500 kDa) and HA nanoparticle (1500 kDa) mixtures: storage modulus over frequency range. Increasing nanoparticle concentration decreased storage modulus.

FIG. 48 shows viscoelasticity of HA polymer (1500 kDa) and HA nanoparticle (1500 kDa) mixtures: loss modulus over frequency range. Increasing nanoparticle concentration decreased loss modulus.

FIG. 49 shows viscoelasticity of HA polymer (1500 kDa) and HA nanoparticle (1500 kDa) mixtures: complex modulus over frequency range. Increasing nanoparticle concentration decreased complex modulus.

FIG. 50 shows viscoelasticity of HA polymer/nanoparticle mixtures: storage modulus over frequency range. Storage modulus of the samples composed of 17 kDa HA nanoparticles were greater than the storage modulus of the samples made from 1500 kDa HA nanoparticles at all polymer: nanoparticle ratios.

FIG. 51 shows viscoelasticity of HA polymer/nanoparticle mixtures: loss modulus over frequency range. Loss modulus of the samples composed of 17 kDa HA nanoparticles were greater than the loss modulus of the samples made from 1500 kDa HA nanoparticles at all polymer: nanoparticle ratios.

FIG. 52 shows viscoelasticity of HA polymer/nanoparticle mixtures: complex modulus over frequency range. Complex modulus of the samples composed of 17 kDa HA nanoparticles were greater than the complex modulus of the samples made from 1500 kDa HA nanoparticles at all polymer: nanoparticle ratios.

FIG. 53 shows viscoelasticity of HA nanoparticle suspensions at different nanoparticle concentrations: storage modulus over frequency range. Increasing nanoparticle concentration increased storage modulus.

FIG. 54 shows viscoelasticity of HA nanoparticle suspensions at different nanoparticle concentrations: loss modulus over frequency range. Increasing nanoparticle concentration increased loss modulus.

FIG. 55 shows viscoelasticity of HA nanoparticle suspensions at different nanoparticle concentrations: complex modulus over frequency range. Increasing nanoparticle concentration increased complex modulus.

The patent or application file contains at least one drawing executed in color. Copies of this patent or patent application publication with color drawing(s) will be provided by the Office upon request and payment of the necessary fee.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION

The present disclosure generally relates to polymers. More particularly, the present disclosure relates to hyaluronic acid particles and their applications.

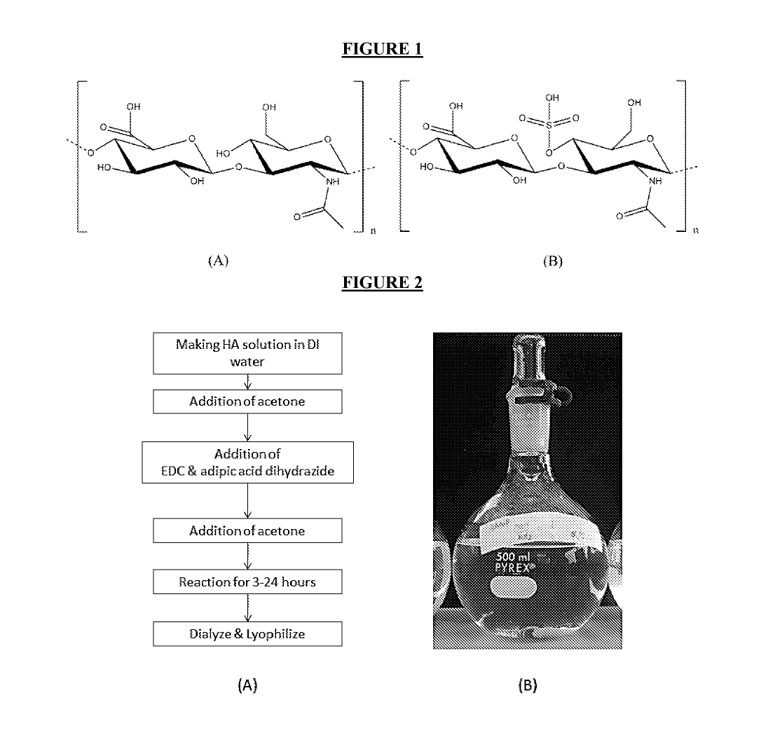

Hyaluronic acid (HA), also named hyaluronan, is a high molecular weight (10⁵-10⁷ Da) naturally occurring biodegradable polymer. HA is an unbranched non-sulfated glycosaminoglycan (GAG) composed of repeating disaccharides (β-1,4-D-glucuronic acid (known as uronic acid) and β-1,3-N-acetyl-D-glucosamide) (FIG. 1). HA can include several thousand sugar molecules in the backbone. HA is a polyanion that can self-associate and that can also bind water molecules (when not bound to other molecules) giving it a stiff, viscous quality similar to ‘Jello’.

Structural studies showed that the two sugar molecules, D-glucuronic acid and D-N-acetyl glucosamine, in the HA disaccharide structure are connected together through alternative beta-1,4 and beta-1,3 glycosidic bonds (FIG. 1). The hyaluronic acid backbone is stiffened in physiological solution via a combination of internal hydrogen bonds, interactions with solvents, and the chemical structure of the disaccharide. HA molecular investigations suggested that the axial hydrogen atoms form a non-polar face (relatively hydrophobic) and the equatorial side chains form a more polar face (hydrophilic) led to a twisted ribbon structure for HA called a coiled structure.

HA's structural characteristics hinge on this random coiled structure in solution. At very low concentrations, chains entangle each other, leading to a mild viscosity (molecular weight dependent). On the other hand, HA solutions at higher concentrations have a higher than expected viscosity due to greater HA chain entanglement that is shear-dependent. For instance, a 1% solution of high molecular weight HA (Mw>˜1000 kDa) can behave like jelly, but when shear stress is applied it will easily shear thin and can be administered via a thin needle. As such, HA is known as a “pseudo-plastic” material. This extraordinary rheological property (concentration and molecular weight dependent) of HA solutions has made HA ideal for lubrication in biomedical applications.

In addition to the unique viscosity of HA, viscoelasticity is another characteristic of HA resulting from entanglement and self-association of HA random coils in solution. It was suggested that the molecular self-association of HA occurs by forming anti-parallel double helices, bundles, and ropes. Further experiments verified that HA chain-chain association occurred in solution. Moreover, studies proposed that hydrogen bonding between adjacent saccharides occurred alongside mutual electrostatic repulsion between carboxyl groups, thus stiffening HA. Viscoelasticity of HA can be tied to these molecular interactions which are also dependent on concentration and molecular weight.

Since hyaluronic acid is one of the main components of body tissues, its potential for tissue engineering applications has been highly touted. HA is highly soluble at room temperature and has a high rate of elimination and turnover depending on its molecular weight and location in the body. Each of these properties could be a barrier for HA scaffold fabrication and structural integrity. To overcome these limitations, crosslinking of hyaluronic acid has been proposed. Currently, water-soluble carbodiimide crosslinking, polyvalent hyadrazide crosslinking, divinyl sulfone crosslinking, disulfide crosslinking, and photocrosslinking of hydrogels through glycidyl methacrylate-HA conjugation have been introduced for tissue engineering applications of HA. Chemical crosslinking of HA provides the ability to combine the desirable biological and mechanical properties, even for bone or cartilage tissue engineering. Moreover, crosslinking extends the HA degradation process in vivo and provides long-term stability. Crosslinking HA at various densities has been used for multiple applications including orthopedics, cardiovascular medicine, and dermatology.

Studies have suggested positive results for cell growth on photocrosslinked HA networks incorporated with chondrocytes. Chondrocytes within the HA hydrogel retained viability and were able to generate cartilage within the porous network. This type of photopolymerization has also been used in heart valve applications to mimic cardiac valve development. HA has also been combined with other polymers such as polypyrrole to develop multifunctional copolymers. HA functionalized with polypyrrole is electronically conductive and supports cell growth. This copolymerization could have potential for tissue engineering applications. Benzyl derivatives of HA is another category of polymeric scaffolds used for tissue engineering of cartilage with predictable degradation rates. Studies on these derivatives suggested the potential of benzyl esters of HA as a delivery scaffold for chondrocytes in cartilage tissue engineering. Since HA is a biocompatible natural polymer, development of scaffolds based on HA appears to be suitable for surfaces contacting blood. For example, HA crosslinked with divinyl sulfone (DVS) in the presence of ultraviolet light has been suggested to develop “non-activating” surfaces for cell adhesion in heart valve tissue engineering.

Auto-crosslinked and in situ crosslinked HA hydrogels are another category of crosslinking used for tissue engineering. The requirement for surgical implantation is the major limitation of most scaffolds used for tissue engineering. Application of HA that crosslinks after injection has been introduced for three main reasons. First, the injectable HA could be filled into any desirable shape and crosslinked in situ. Second, crosslinkable HA may adhere to the native tissue resulting in mechanical or chemical interlocking and a cohesive scaffold-tissue interface. Third, injection and laparoscopic methods can be used to reduce the invasiveness of the surgical procedure. Studies showed that in situ crosslinked HA hydrogel using adipic acid dihydrazide and aldehyde chemistry could form a flexible hydrogel in situ upon mixing. In another study, poly(lactic-co-glycolic acid) nanoparticles were mixed with HA of similar chemistry to develop an in situ crosslinkable system with drug delivery potential. Although such in situ crosslinking has been shown to form flexible hydrogels with reasonable mechanical properties, potential toxicity of the reactions used in these techniques are still an important issue to consider.

The present disclosure addresses improvements to applications of HA in areas including but not limited to, tissue engineering, dermal filling, and viscosupplementation. In each application, difficulties such as potential toxicity of in situ crosslinking techniques, high viscosity of HA solutions, and rapid elimination have been raised as limitations to develop biomedical products from HA. Nanotechnology may provide an approach to resolve these limitations.

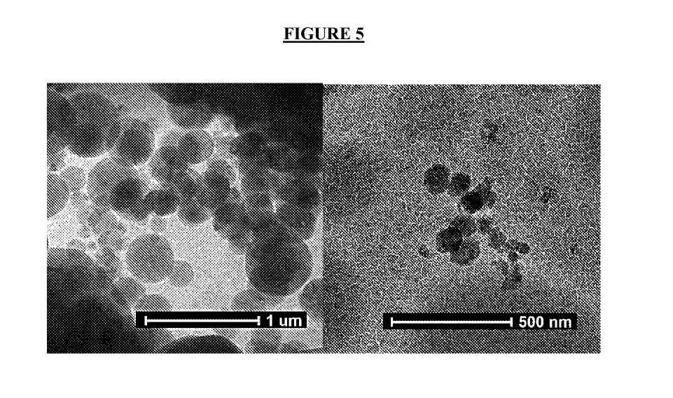

Fabrication of particles from polymers can affect bulk properties, including but not limited to, physical, mechanical, and rheological, of ensuing material. Accordingly, the present disclosure provides a particle fabrication technique for HA. The present disclosure further provides the use of HA particles to modify viscosity and viscoelasticity of HA solutions or suspensions. In one embodiment, the present disclosure provides for a method to fabricate “hairy” HA particles. As used herein, the term “hairy” is used to describe a particle with a plurality of free polymer chains extending from the surface of the particle such that the polymer chains on the surface are capable of association with other polymers or with polymer chains on the surface of other particles. The free polymer chains extending from the surface of the particle comprise free —COOH groups. The presence of free —COOH groups on the polymer chains that extend from the surface of the particle does not mean, for example, that other portions of the chain have not participated in a crosslinking reaction. One representation of a “hairy” particle is illustrated in FIG. 4.

In one embodiment, the present disclosure provides a method comprising providing a hyaluronic acid polymer, wherein the hyaluronic acid polymer has a molecular weight in the range of from about 5 kDa to about 100 kDa; crosslinking the hyaluronic acid polymer with adipic acid dihydrazide crosslinker in a mixture comprising an 1-Ethyl-3-[3-dimethylaminopropyl]carbodiimide hydrochloride activator, water, and acetone, wherein molar reactive site ratio is greater than about 1:1.isolating hyaluronic acid particles from the mixture, wherein at least a portion of the hyaluronic acid particles comprise a plurality of free polymer chains extending from a surface of the particle.

The molecular weight of the HA polymer is generally within the range of from about 5 kDa to about 100 kDa. In certain embodiments, the molecular weight of the HA polymer is in the range of about 5 kDa to about 10 kDa, in certain embodiments, from about 10 kDa to about 15 kDa, in certain embodiments, from about 15 kDa to about 20 kDa, in certain embodiments, from about 20 kDa to about 25 kDa, in certain embodiments, from about 25 kDa to about 30 kDa, in certain embodiments, from about 30 kDa to about 35 kDa, in certain embodiments, from about 35 kDa to about 40 kDa, in certain embodiments, the molecular weight is in the range of from about 40 kDa to about 45 kDa, in certain embodiments, from about 45 kDa to about 50 kDa, in certain embodiments, from about 50 kDa to about 55 kDa, in certain embodiments, from about 55 kDa to about 60 kDa, in certain embodiments, from about 60 kDa to about 65 kDa, in certain embodiments, from about 65 kDa to about 70 kDa, in certain embodiments, from about 70 kDa to about 75 kDa, in certain embodiments, from about 75 kDa to about 80 kDa, in certain embodiments, from about 80 kDa to about 85 kDa, in certain embodiments, from about 85 kDa to about 90 kDa, in certain embodiments from about 90 kDa to about 95 kDa, in certain embodiments, from about 95 kDa to about 100 kDa. In certain embodiments, the molecular weight of the HA polymer may be less than 30 kDa. In certain embodiments, the molecular weight of the HA polymer may be from about 10 kDa to about 20 kDa.

The molecular weight of the HA polymer is one factor that determines the resulting properties of the HA particles. In particular, the size of the resulting HA particles and whether the resulting HA particles comprise a plurality of free polymer chains extending from the surface of the particle may be dependent on the molecular weight of the HA polymer. For HA polymers with molecular weights of less than 100 kDa, due to the short length of the polymer, the chance of intramolecular crosslinking during the fabrication of nanoparticles may be less than the chance of intermolecular crosslinking. On the other hand, the chance of intramolecular crosslinking of HA polymers of higher molecular weight may be greater due to its longer polymer chain length, more coiled structure, and greater self-association compared to HA polymers of lower molecular weight (See FIG. 4). Thus, this may provide a packed structure for nanoparticles made from higher molecular weight. HA polymers also resulting in smaller particle size compared to nanoparticles made from lower molecular weight HA polymers. In addition, more “dangling” or free polymer chains may be available on the surface of nanoparticles made from lower molecular weight HA polymers due to the greater chance of intermolecular crosslinking compared to nanoparticles made from higher molecular weight HA polymers (FIG. 4). The presence of dangling or free polymer chains on the nanoparticles may affect particle-particle and particle-polymer interactions.

The molar reactive site ratio, as used herein, is the ratio of COOH groups on the HA polymer to NH₂ groups on the crosslinker. (—COOH:—NH₂). The molar reactive site ratio is also a factor that determines the properties of the resulting HA particles. HA particles that are produced using a molar reactive site ratio in which the amount of primary amine groups is higher than the amount of COOH groups tend to have larger particle sizes and lack the free polymer chains on the surface, which may be due to the molar excess of crosslinker resulting in a higher probability of polymer-HA particle or HA particle-HA particle reaction. Thus, as the crosslinker content decreases in a reaction mixture, the HA particles have a higher likelihood of containing free polymer chains on their surface. Thus, it is preferable in the methods of the present disclosure to have a molar reactive site ratio in which the amount of —COOH groups is greater than or equal to the amount of —NH₂ groups. In certain embodiments, the molar reactive site ratio may be greater than or equal to 1:1. In certain embodiments, the reactive site ratio may be in the range of from about 1:1 to about 2:1; in certain embodiments, the reactive site ratio may be in the range of from about 1:1 to about 3:1, in certain embodiments, the reactive site ratio may be in the range of from about 2:1 to about 3:1. In certain embodiments, the molar reactive site ratio may be greater than 3:1. The “hairy” HA particles that are produced according to the methods of the present disclosure are dependent on both the molecular weight of the HA polymer and the molar reactive site ratio. One of ordinary skill in the art would understand that an amount of crosslinker may be used in a reaction greater than the amount to achieve the desired molar reactive site ratio because not all of the crosslinker containing primary amine groups will react with the HA polymer.

In certain embodiments, the present disclosure allows for the application of HA particles to develop materials for tissue engineering, dermal filling, and viscosupplementation. In certain embodiment, the HA particles may be HA microparticles. In certain embodiments, the HA particles may be HA nanoparticles. As used herein, HA nanoparticles are nanometer scaled particles of HA. As used herein, colloidal gels are stable 3-D networks made from particles. In contrast to colloidal gels, colloidal suspensions, as used herein, easily flow by applying shear force.

In certain embodiments, the colloidal gels of the present disclosure can be used to develop biodegradable scaffolds for tissue engineering. Crosslinking is a common technique employed for HA scaffold fabrication. Almost all of the crosslinking reactions used for this purpose are toxic to cells; however, the desired mechanical properties can be achieved for tissue engineering applications. In addition to that, injectability of viscous HA is another desired property that can help physicians to fill a defect site without invasive surgery. It is an object of the present disclosure to develop a new type of colloidal material based on HA particles, which can be used as a self-assembled scaffold without utilizing chemical reactions. The injectability of the colloidal material and its behavior under shear stress was evaluated to determine applicability as an injectable scaffold for tissue engineering.

Colloidal gels have been suggested as tissue engineering scaffolds, drug delivery systems, and biosensors. Interparticle interactions including electrostatic interactions, van der Waals forces, and steric hindrance enable the internal cohesion of colloidal gels. Applying shear force can disrupt these interactions allowing the colloidal gels to flow. To stabilize the structure of colloid-containing materials, chemical reactions such as in situ crosslinking have been reported, although the application of these reactions are limited due to toxicity. Advanced techniques need to be developed to stabilize the mechanical properties or to enhance dynamic properties of colloidal gels while maintaining compatibility with tissues.

The fabrication of moldable colloidal gels has already been reported for tissue engineering applications, but few reports utilize particles made from biomaterials commonly used as tissue engineering scaffolds. According to the present disclosure, a novel colloidal gel based on HA particles was explored as a means to form stable 3-D networks. As an alternative to inter-particle electrostatic interactions, other properties of polymers such as physical entanglement may occur between ‘self-associating’ polymeric particles. In certain embodiments, the particles may be chondroitin sulfate particles. One advantage of the colloidal gels of the present disclosure is that the gels can be formed simply by mixing particles in water, without the need for a chemical reaction, which is generally required for the formation of hyaluronic acid hydrogels.

In another embodiment, HA particles may also be used to enhance the properties of dermal fillers, including, but not limited to, increasing the injectability of dermal fillers. Hyaluronic acid has been approved by the Food and Drug Administration (FDA) as a dermal filler. In 2006, cosmetic injections of HA were known to be the second most popular non-surgical procedure for women and the third most popular procedure for men. HA has a very short half-life and, therefore, is chemically crosslinked to extend duration as a dermal filler. HA is not involved in the structure of collagen and does not enhance tissue, the shortage of HA, in aged skin, but simply works by augmenting volume. The development of dermal filler products with enhanced injectability and longer duration is desired. It seems an ideal dermal filler should be temporary but long-lasting, having minimum side effects and no allergenic effect, easy to administer, having minimum pain or no pain upon injection, and a reasonable cost for both the physician and the patient.

HA based dermal fillers are one of the most popular for temporary treatment within a duration of up to 12 months. These crosslinked HA solutions are viscous and difficult to administer with fine needles. Modifying these supplements with HA particles, according to the present disclosure, may extend the residency of the HA in vivo and increase the treatment duration to more than 12 months. Moreover, particles may also help to reduce the viscosity of these solutions for easy injection via fine needles. The present disclosure provides for HA polymer solutions with HA particles as an additive to modify the viscosity and the concentration of HA in solution. The desired concentration of HA in solution will depend on the intended application of the final HA solution. Moreover, the modified HA polymer solutions of the present disclosure may be used, among other things, as a dermal filler.

In other embodiments, the composition and methods of the present disclosure may be used in the treatment of osteoarthritis. Osteoarthritis (OA) is the most common disease associated with aging, affecting approximately 33 million Americans with about 70% of these individuals aged 65 and over. OA is characterized by the slow degradation of cartilage, pain, and increasing disability. The disease can have an impact on several aspects of a patient's life, including functional and social activities. Current pharmacological therapies target palliation of pain and include analgesics (i.e. acetaminophen, cyclooxygenase-2-specific inhibitors, non-steroidal anti-inflammatory drugs, tramadol, opioids), intra-articular therapies (glucocorticoids and hyaluronan), and topical treatments (i.e. capsaicin, methylsalicylate). If none of these therapies work, surgical joint replacement is the last option, which is costly and highly invasive.

Synovial spaces are the cavities of the joints that facilitate movement of adjacent bones. Synovial spaces are formed by a surface of cartilage, synovium, and synovial fluid. The synovial fluid is a clear, colorless or sometimes yellowish liquid secreted into the joint cavity by the synovium. The synovial fluid volume is approximately 2 mL in normal human knee joints and contains electrolytes, low molecular weight organic molecules, and macromolecules such as glycosaminoglycans (GAGs). GAGs present in the synovial fluid are chondrotin-4-sulfate (2%), with the remaining 98% made up of HA.

The mechanical function of the synovial fluid can be attributed to its rheological properties, more specifically its viscoelastic properties. Synovial fluid viscoelasticity may be ascribed to the concentration, molecular weight, and molecular weight distribution, and to the physical and non-covalent interactions within the HA molecule as well as with other molecules such as proteins and ions. HA molecules overlap and interact through association, which may involve physical entanglement or temporary crosslinking interactions with ions and proteins at physiological conditions. These interactions, which are dependent on HA molecular weight and concentration, determine the formation of the transient network structure that is responsible for the viscoelasticity of synovial fluid. In OA, HA loses these functionalities as a result of reduced HA molecular weight and concentration; thus, decreasing the viscoelastic properties of synovial fluid.

After damage or aging, synovial fluid cannot provide the required viscoelastic response to compression and tangential forces arising in everyday life, allowing cartilage-cartilage contact and increasing wear of the joint surface. Intra-articular treatment with HA and hylans (uncrosslinked HA and crosslinked HA, respectively) has recently been accepted as a common therapy for reducing pain associated with OA. Currently, FDA-approved products such as Hyalgan® (HA), Orthovisc® (HA) and Synvisc® (hylan GF 20) are available as viscosupplements for intra-synovial injection in osteoarthritis treatment Table (1-5).

Several clinical trials have demonstrated the efficacy and tolerability of intra-articular HA for the treatment of pain associated with OA. These studies have shown three injections of Synvisc® (crosslinked HA) can provide relief of knee pain up to 6 months. A competing product, Hyalgan® (sodium hyaluronate solution), requires 6 injections to reach the same efficacy of Synvisc®. While Synvisc® was shown to be more efficient in reducing pain, its structure (high molecular weight HA due to crosslinking) has made this difficult to inject (Table 1-6). Unlike Synvisc®, Hyalgan® has a lower viscosity, making injection easier, but Hyalgan® is not as effective as Synvisc® due to lower viscoelasticity. Moreover, Orthovisc®, one of the viscosupplements with the highest HA concentration, has lower viscosity than Synvisc® but it is not reported to be as effective as Synvisc®. Though crosslinking may increase the performance of viscosupplements thereby extending the treatment duration, crosslinking increases the viscosity of these viscosupplements making them difficult to inject.

To enhance injectivity, multiple studies have been conducted and several low viscosity products are available on the market. Using HA with lower molecular weights has been the primary means to reduce viscosity of vicosupplements; however, low molecular weight HA does not provide a sufficient therapeutic effect. Hyalgan® is one of these low molecular weight, uncrosslinked HA viscosupplements. More injections of Hyalgan® are required per treatment course to achieve a reasonable therapeutic effect (Table 1-5). On the other hand, treatment with crosslinked HA viscosupplements such as Synvisc® requires fewer injections per treatment course but their high viscosity impedes injection. This creates a need for development of products with enhanced injectability and yet viscoelastic characteristics.

TABLE (1-5)
Some of the HA viscosupplements available in the North
American market.

| Brand name | Molecular | Approved | | Amount per | |
|---|---|---|---|---|---|
| (Generic name) | weight (kDa) | dosing* | | injection (mL) | Approved indications |
| Durolane ® | 1000 | 1 | injection | 3 | Knee or hip, mild or moderate |
| (Sodium hyaluronate, 2%) | | | | | |
| Fermathron ® | 1000 | 3-5 | injections | 2 | Knee, mild or moderate |
| (Sodium hyaluronate, 1%) | | | | | |
| Hyalgan ® | 500-730 | 3-5 | injections | 2 | Knee, shoulder, or hip |
| (Sodium hyaluronate, 1%) | | | | | |
| NeoVisc ® | 1000 | 3-5 | injections | 2 | Synovial fluid replacement |
| (Sodium hyaluronate, 1%) | | | | | following arthrocentesis |
| Orthovisc ® | 1000-2000 | 3 | injections | 2 | Knee |
| (Sodium hyaluronate, 1.4%) | | | | | |
| Ostenil ® | 1000-2000 | 3 | injections | 2 | Degenerative or traumatic |
| (Sodium hyaluronate, 1%) | | | | | synovial joint disorders, |
| | | | | | including knee joint |
| Supartz ® | 620-1170 | 3-5 | injections | 2.5 | Knee nonresponsive to |
| (Sodium hyaluronate, 1%) | | | | | conservative therapy |
| Suplasyn ® | 500-730 | 3-6 | injections | 2 | Synovial fluid replacement |
| (Sodium hyaluronate, 1%) | | | | | following arthrocentesis |
| Synvisc ® | 6000-7000 | 3 | injections | 2 | Knee nonresponsive to 0.8% |
| (Hylan G-F 20; Crosslinked | | | | | conservative therapy |
| HA) | | | | | |

*The number of weekly intra-articular injections per treatment course, excluding Durolane ®, which is given as a single injection.

TABLE (1-6)
Properties of Hyalgan ® and Synvisc ®. Data were adapted from
Hyalgan ® product information, Orthovisc ® product information,
Synvisc ® product information, and references.

| | Molecular | Viscoelastic properties | | | |
|---|---|---|---|---|---|
| Brand | weight | Elasticity (G′) | Viscosity (G″) | Number of | Duration of |
| name | (kDa) | (Pa) at 2.5 Hz | (Pa) at 2.5 Hz | injections | pain relief |
| Hyalgan ® | 500-730 | 0.6 | 3 | 3-5 | 6 months |
| (Uncrosslinked) | | | | | |
| Orthovisc ® | 1000-2000 | 60 | 46 | 3 | 6 months |
| (Uncrosslinked) | | | | | |
| Synvisc ® | 6000-7000 | 111 ± 13 | 25 ± 2 | 3 | 6 months |
| (Crosslinked) | | | | | |

In another embodiment of the present disclosure, HA particles may be employed to modify viscosupplements used for osteoarthritis treatment. Currently used viscosupplements are not formulated with HA particles. High viscosity of viscosupplements such as Synvisc® has always been an issue. According to one embodiment, the viscosity of HA solutions by using HA particles was reduced. Viscoelasticity, another characteristic of HA solutions (and suspensions), was evaluated to determine the effect of particles in HA solutions (and suspensions). The addition of HA particles in viscosupplements, which includes but are not limited to HA polymer solutions, manipulates solution viscosity and viscoelasticity. Physical interactions between particles and polymer in solution depends on the molecular weight of the HA used for fabricating the particles. For example, using a 17 kDa HA can increase the viscosity and yield point of the HA acid solutions.

In other embodiments, the colloidal gels of the present disclosure can be used for drug delivery and targeting. In other embodiments, the colloidal gels of the present disclosure can be used to the extend the performance of commercially available viscosupplements and dermal fillers.

To facilitate a better understanding of the present invention, the following examples of certain aspects of some embodiments are given. In no way should the following examples be read to limit, or define, the entire scope of the invention.

### EXAMPLES

### Example 1

### Nanoparticle Fabrication

One of the natural polymers used for nanoparticle fabrication is hyaluronic acid (HA). Several methods have been proposed to fabricate HA nanoparticles. Electrostatic interactions with positively charged polymers such as chitosan or biological components such as proteins have been shown to form nanoparticles. Changing suspension conditions such as pH and ionic strength can dissociate the polyelectrolyte systems. Therefore, these types of nanoparticles may only be stable under specific conditions in which the particles are formed. Studies have utilized electrostatic interactions to synthesize nanoparticles via complexation of HA with cationic polymers and therapeutic agents for delivery of anti-tumor drugs, treatment of asthma, and active tumor targeting. Chemical crosslinking has also been suggested to fabricate hydrophobic core-hydrophilic shell HA nanoparticles (Yang et al., 2011) Unlike electrostatic interactions, structurally stable HA nanoparticles can be synthesized via chemical crosslinking techniques. Not many reports are available for HA nanoparticle fabrication using chemical crosslinking. Techniques including water-in-oil-emulsion processes, spray drying, and solvent evaporation have also been reported to make micron-scale HA particles (Kyyronen et al., 1992); however, these studies showed that completely removing oil and surfactant from the HA particles is difficult. Controlling the size distribution of particles by spray drying can also be difficult (Hu et al., 2004). Therefore, the development of HA nanoparticle fabrication methods which produce relatively uniform and stable colloids without the use of oil and surfactant is desirable (Hu et al., 2004).

A technique free from oil and surfactant by Hu et al. was used to fabricate HA nanoparticles (Hu et al., 2004). Chemical crosslinking based on carbodiimide chemistry was used to synthesize nanoparticles from HA polymer. Besides hyaluronic acid, chondroitin sulfate (CS), another naturally occurring glycosaminoglycan (GAG), was also selected to make nanoparticles in this study. Dynamic light scattering was employed to evaluate the effect of polymer type (hyaluronic acid and chondroitin sulfate), polymer concentration, HA molecular weight, reaction time, and the ratio between polymer to crosslinker on the size and charge of nanoparticles. Then, cryo-transmission electron microscopy was used to understand the morphology of nanoparticles. Fourier transform infrared spectroscopy (FTIR) was also employed to confirm the crosslinking reaction. These synthesized nanoparticles were used to understand the possibility of colloidal gel fabrication and the effect of nanoparticles on the properties of HA suspensions.

Polymers of hyaluronic acid (HA) sodium salt (HA with different molecular weights of 17 kDa, 741 kDa, and 1500 kDa) were purchased from Lifecore Biomedical (Chaska, Minn.). 1-Ethyl-3-[3-dimethylaminopropyl]carbodiimide hydrochloride (EDC) was purchased from Thermo Scientific (Rockford, Ill.). Acetone was obtained from Fisher Scientific (Fair Lawn, N.J.). Chondroitin sulfate sodium salt from bovine cartilage (Mw 30 kDa), adipic acid dihydrazide, and trinitrobenzene sulfonic acid (TNBS) (˜1 M in H₂O) were purchased from Sigma-Aldrich (St. Louis, Mo.). Dialysis membrane (Regenerated Cellulose (RC), MWCO 50,000) was obtained from Spectrum Laboratory Products Inc. (Rancho Dominguez, Calif., USA).

Nanoparticle Fabrication Method

Nanoparticles were fabricated by adapting a technique described by Hu et al. (2004). Nanoparticles synthesized in by this technique were used in later experiments to understand the possibility of colloidal gel fabrication and the effect of nanoparticles on the properties of HA suspensions.

In this method, nanoparticles were synthesized based on crosslinking polymer chains through their carboxyl groups via carbodiimide chemistry. Briefly, aqueous solution of polymer (HA or CS) was prepared by mixing polymer in deionized water (2.5 mg/mL, 80 mL) in a 500 mL round flask. Then, 136 mL acetone was added to the flask and stirred for 15 minutes (500 rpm) to make sure all the components in the solution were well dispersed. To avoid acetone evaporation, the flask was sealed properly. EDC (80 mg) and adipic acid dihydrazide (40 mg) were dissolved in 2 mL of deionized water and added to the flask. After mixing the solution for 30 minutes, another 131 mL acetone was added to the solution and stirring was continued for another 3 hours (Final HA concentration˜0.57 mg/mL). Then, the reaction was stopped by dialysis of the solution against deionized water using dialysis membrane (Regenerated Cellulose (RC), MWCO 50,000). Finally, the nanoparticles were freeze dried and dry powder was stored at −20° C. (FIG. 2).

EDC activates carboxyl groups available on HA (or CS) and provides reactive intermediates (O-acylisourea derivatives, extremely short-lived) which react with two primary amines of adipic acid dihydrazide forming a peptide bond and resulting in the neighboring HA (or CS) chains being chemically crosslinked (FIG. 3). With the first addition of acetone, there were no nanoparticles formed in a control experiment. After the second addition of acetone, the volume ratio of acetone/water reached to approximately 269/80 (the ratio range to form HA (or CS) nanoparticles is between 200/80 to 300/80) (Hu et al., 2004). This acetone/water ratio was reported to break the strong hydrogen bonding between HA (or CS) chains and HA-water molecules (or CS-water molecules) to release carboxyl groups for the crosslinking reaction. After the consumption of carboxyl groups by adipic acid dihydrazide, the crosslinked HA (or CS) polymer chains become less soluble (hydrophilic) and were suggested to transform from coils to globules the resulting solution turns from clear to light blue, indicating nanoparticle formation (FIG. 2) (Hu et al., 2004).

Particle Size and Zeta Potential Characterization

Particle size and zeta potential were measured using a ZetaPALS dynamic light scattering instrument (Brookhaven, USA) after dispersion of freeze dried nanoparticles in deionized water (2 mg/mL). A cryo-transmission electron microscope (FEI field emission transmission electron microscope, Tecnai™ G2 at 200 kV) was employed for morphological characterization. Cryo-TEM samples were prepared using a Vitrobot™ (FEI), a PC-controlled robot for sample preparation. Substratek™ grids with 2-3 nm platinum coating on 400 square mesh gold grids were used (Ted Pella Inc., California) for sample preparation. First, nanoparticles were dispersed in deionized water at 2 mg/mL concentration. Then, 3 μl of nanoparticle suspension was placed on the grid, blotted to reduce film thickness (3 seconds blot time), and vitrified in liquid ethane. Finally, the grid was transferred to liquid nitrogen for storage and imaging was performed after placing the prepared grid into a cryo sample holder filled with liquid nitrogen.

Characterization of Bond Formation Via FTIR

To confirm peptide bond formation between HA carboxyl groups and adipic acid dihydrazide amine groups, a Spectrum 100 FTIR Spectrometer was used (PerkinElmer, Inc., Massachusetts). The FTIR spectra for the starting polymer and fabricated nanoparticles were compared. Adipic acid dihydrazide was also used to identify its related peaks.

Evaluation of Crosslinker Consumption

Analysis of fabricated nanoparticles with trinitrobenzene sulfonic acid (TNBS) allows quantification unreacted adipic acid dihydrazide. TNBS is known to react to amines, hydrazines, and hydrazides creating a stable trinitrophenyl complex. The trinitrophenyl complex of adipic dihydrazide has maximum absorbance at λ=334 nm, which can be detected via spectrophotometry.

Briefly, nanoparticles made from 17 kDa HA were dispersed in deionized water at 5 mg/mL concentration. A 17 kDa HA polymer solution at the same concentration was also prepared as a negative control sample. Adipic acid dihydrazide (0.833 mg/mL) was also added to separate nanoparticle suspension and polymer solution (5 mg/mL) to make positive controls. To one mL of nanoparticle suspension, negative control, or positive controls, 5 μL TNBS solution was added. Then, the samples were incubated at 37° C. for one hour to let TNBS react with primary amines of adipic acid dihyadrazide resulting in trinitrophenyl complex formation. Finally, 100 μL of each sample was transferred into three wells of a 96-well cell culture plate and the absorbance of trinitrophenyl complex of adipic acid dihydrazide was measured at λ=334 nm using a microtiter plate reader (SpectraMax, M25, Molecular Devices Corp., CA). HA nanoparticle suspension and polymer solution (without TNBS) was also used to quantify the absorbance of nanoparticles at a similar wavelength.

Statistical Analysis

Statistical analysis was performed using one-way analysis of variance (ANOVA). To evaluate the significance of differences, Newman-Keuls was used as a post-hoc test. A value of p<0.05 was accepted as significant in all cases.

Nanoparticle Fabrication and the Effect of Process Parameters on Size and Charge of the Particles

Hyaluronic acid and chondroitin sulfate nanoparticles were successfully fabricated. It appears the fabrication of chondroitin sulfate nanoparticles and nanoparticles made from different molecular weights of HA has not been reported previously. This technique could enable fabrication of nanoparticles from other polymers if the carobodiimide chemistry is transferable.

The effect of polymer concentration (0.28 mg/mL, 0.57 mg/mL, and 2.28 mg/mL) on the size and charge of HA (17 kDa) and CS nanoparticles was evaluated (Table 2-1).

TABLE (2-1)
Effect of polymer concentration on size and charge of HA and CS
nanoparticles. Polydispersity values were less than 0.22. Data
represent the mean ± SD (n = 3).

| | Hyaluronic | | Chondroitin | |
|---|---|---|---|---|
| | acid (17 kDa) | | sulphate | |
| Polymer | | Zeta | | Zeta |
| concentration | Particle size | potential | Particle size | potential |
| (mg/mL) | (nm) | (mV) | (nm) | (mV) |
| 0.28 | 167 ± 15.5 | −31 ± 1.1 | 177 ± 3.1* | −50 ± 0.3 |
| 0.57 | 188 ± 23.9* | −32 ± 2.9 | 282 ± 1.1 | −52 ± 1 |
| 2.28 | 564 ± 27.2** | −36 ± 0.7 | 449 ± 2.1** | −49 ± 0.1 |

*p < 0.005 comparing 0.57 mg/mL to 0.28 mg/mL (HA data) and comparing 0.28 mg/mL to 0.57 mg/mL (CS data).
**p < 0.005 comparing 2.28 mg/mL to 0.28 mg/mL (HA data) and comparing 2.28 mg/mL to 0.57 mg/mL (CS data).

Increasing polymer concentration increased particle size for both hyaluronic acid (HA Mw=17 kDa) and chondroitin sulfate (CS Mw˜30 kDa). The size of HA nanoparticles at 2.28 mg/mL polymer concentration was significantly greater than the size of the nanoparticles at 0.28 mg/mL and 0.57 mg/mL polymer concentration. CS nanoparticles at 0.57 mg/mL polymer concentration were significantly larger than CS nanoparticles at 0.28 mg/mL polymer concentration. The data suggested that increasing polymer concentration increases particle size. This increase was shown to be independent of polymer type and might be due to more intermolecular crosslinking at higher concentrations resulting in fabrication of larger particles. CS nanoparticles showed greater negative charge values compared to HA nanoparticles. This is likely be due to the presence of functional groups available on CS and ionization of these groups in suspension resulting in more negative charge for CS nanoparticles. Finally, these results indicated that the charge of the nanoparticles were not significantly different at different polymer concentrations.

Molecular weight of HA used for nanoparticle fabrication influenced the size of the nanoparticles. HA with three different molecular weights (17 kDa, 741 kDa, and 1500 kDa) was used to evaluate the effect of polymer molecular weight on particle size and charge (Table 2-2).

TABLE (2-2)
Effect of HA molecular weight on size and charge of HA
nanoparticles. Polydispersity values were less than 0.21.
Data represent the mean ± SD (n = 3).

| HA molecular | | |
|---|---|---|
| weight | Particle size (nm) | Zeta potential (mV) |
| 17 kDa | 188 ± 23.9* | −32 ± 2.9 |
| 741 kDa | 78 ± 0.7** | −21 ± 0.5 |
| 1500 kDa | 87 ± 2.5 | −26 ± 0.9 |

* and **p < 0.005 comparing to 1500 kDa HA.

Nanoparticles made from 17 kDa HA were larger than nanoparticles made from 741 kDa HA. Nanoparticles made from 1500 kDa HA were larger than the nanoparticles made from 741 kDa HA but they were significantly smaller than the nanoparticles made from 17 kDa. The zeta potential of the nanoparticles was not dependent on the HA molecular weight and negative charge was observed for all the HA nanoparticles (Table 2-2). Due to the short length of 17 kDa HA, the chance of intramolecular crosslinking during the fabrication of nanoparticles may be less than the chance of intermolecular crosslinking. On the other hand, the chance of intramolecular crosslinking of 1500 kDa HA may be greater due to its long polymer chain length, more coiled structure, and greater self-association compared to 17 kDa HA (FIG. 4). This may provide a packed structure for nanoparticles made from 1500 kDa HA also resulting in smaller particle size compared to nanoparticles made from 17 kDa HA. In addition, more “dangling” chains may be available on the nanoparticles made from 17 kDa HA due to the greater chance of intermolecular crosslinking compared to nanoparticles made from 1500 kDa (FIG. 4). The presence of dangling chains on the nanoparticles may affect particle-particle and particle-polymer interactions.

The effect of process parameters in this nanoparticle fabrication method including reaction time and the molar reactive site ratio (—COOH:—NH₂) were also evaluated (Table 2-3). HA with 17 kDa molecular weight was used for this experiment. Three different reaction times, 3, 10, and 24 hours were selected for nanoparticle fabrication. Three different molar ratio reactive sites (—COOH:—NH₂), 1:2 (excess of primary amine groups), 1:1 (equal molar of carboxyl and primary amine groups), and 2:1 (excess of carboxyl groups) were theoretically calculated for the experiment (Table 2-3).

Increasing reaction time in the formulation with a 1:2 reactive site ratio increased particle size. Normally, the carbodiimide reaction is near complete in two hours. Therefore, the increase in particle size may not be the result of further crosslinking over time. The presence of larger nanoparticles in this formulation at longer reaction times may have been caused by aggregation of nanoparticles. Perhaps this is supported by the lack of change of particle charge at different reaction times. Nanoparticles with a 1:2 reactive site ratio had the largest particle size at all reaction times. That might be due to the molar excess of crosslinker resulting in a higher probability of polymer-nanoparticle or nanoparticle-nanoparticle reaction (Table 2-3).

This study also suggested that the reactive site ratio influenced zeta potential in all reaction times. Increasing the relative amount of primary amine groups in the formulation decreased particle charge. The greater chance for crosslinking at higher molarities of primary amine groups appeared to occupy carboxyl groups and decrease zeta potential resulting in less negative charge for nanoparticles (Table 2-3).

TABLE 2-3
Effect of reaction time and molar reactive site ratio (—COOH:—NH₂) on size and charge of
HA nanoparticles. Polydispersity values were less than 0.31. Data represent the mean ± SD (n = 3).

| | Molar reactive site ratio (—COOH:—NH₂) | | | | | |
|---|---|---|---|---|---|---|
| | 1:2 | | 1:1 | | 2:1 | |
| | | Zeta | | Zeta | | Zeta |
| Reaction | Particle | potential | Particle | potential | Particle | potential |
| time (hours) | size (nm) | (mV) | size (nm) | (mV) | size (nm) | (mV) |
| 3 | 298 ± 7.2 | −22.33 ± 1.2 | 188 ± 23.9 | −32 ± 2.9 | 239 ± 10.1 | −41 ± 0.02 |
| 10 | 350 ± 3.8* | −27.41 ± 1.1 | 209 ± 1.3* | −33.29 ± 1.1 | 225 ± 9.3* | −38 ± 1.3 |
| 24 | 501 ± 1.9** | −19.3 ± 0.61 | 341 ± 0.3 | −36.28 ± 1.0 | 278 ± 7.8 | −41 ± 0.2 |

*p < 0.005 comparing 10 hours data to 3 hours data.
**p < 0.005 comparing 24 hours data to 3 hours data.

Cryo-TEM Imaging

Cryo-transmission electron microscopy was employed to investigate the morphology of HA nanoparticles made from 17 kDa and 1500 kDa HA at 0.57 mg/mL polymer concentration, 1:1 molar reactive site ratio, and three hours reaction time. The images suggested spherical shape for HA nanoparticles made from both 17 kDa and 1500 kDa HA (FIG. 5).

Characterization of Bond Formation Via FTIR

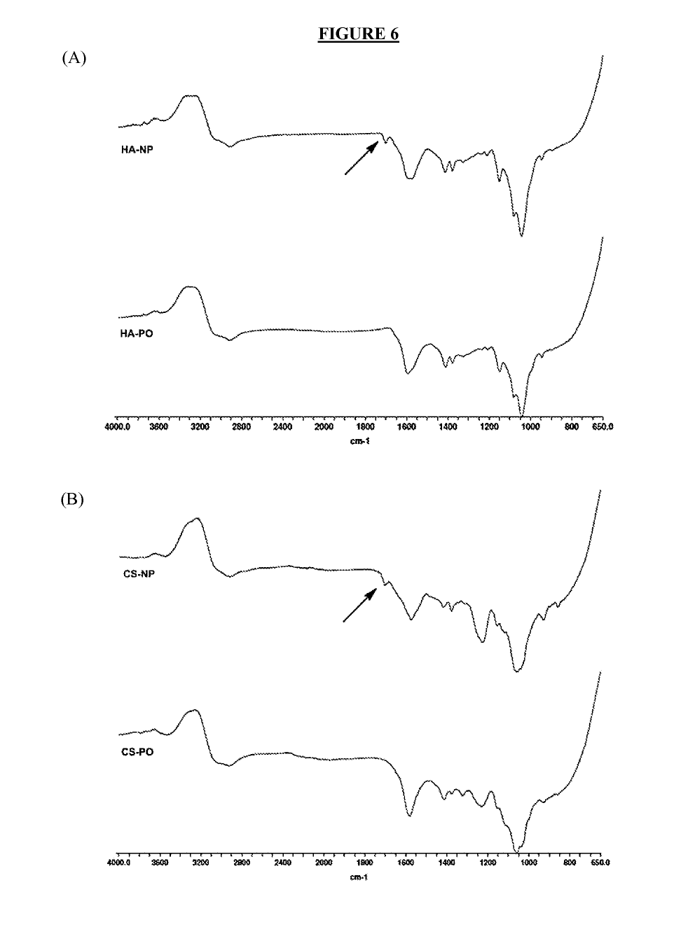

To confirm peptide bond formation between HA carboxyl groups and adipic acid dihydrazide primary amine groups, Fourier transform infrared spectroscopy (FTIR) was employed. The FTIR spectra for the HA nanoparticles (FIG. 6) showed the appearance of a new peak corresponding to the presence of adipic acid dihydrazide (N—N bond) (around 1635 cm⁻¹). This peak appearance was also observed in FTIR spectra of CS nanoparticles (FIG. 6). The presence of these peaks in the FTIR spectrum after purification of nanoparticles confirmed the carbodiimide reaction and peptide bond formation.

Evaluation of Crosslinker Consumption

A TNBS assay was also used to determine the availability of unreacted primary amine groups on the crosslinker after purifying nanoparticles. HA nanoparticles made from 17 kDa HA at 0.57 mg/mL polymer concentration, 1:1 molar reactive site ratio, and three hours reaction time were employed for this experiment. HA polymer was also used as a negative control to compare with the nanoparticles. The TNBS assay revealed that the unreacted primary amine groups on crosslinker were not available in the nanoparticle suspension after purification. Therefore, no subsequent side reactions would occur in the application of HA nanoparticles in colloidal systems, due to elimination of unreacted crosslinker from nanoparticles.

Thus, in these examples, a technique adapted from Hu et al. was used to fabricate nanoparticles. This method was able to successfully synthesize nanoparticles from both hyaluronic acid and chondroitin sulfate using carbodiimide chemistry. Previously, this method has not been employed to fabricate nanoparticles from chondroitin sulfate or from different molecular weights of hyaluronic acid. It was also observed that nanoparticles made from chondroitin sulfate had a more negative charge compared to nanoparticles made from 17 kDa HA. The size and charge of nanoparticles was affected by polymer concentration, polymer molecular weight, reaction time, and the polymer to crosslinker ratio. Increasing polymer concentration increased the particle size; however, HA with higher molecular weights led to smaller particles, presumably with less “dangling” chains. Moreover, FTIR data also suggested the formation of peptide bonds resulting in crosslinking of HA polymer chains via adipic acid dihydrazide. Nanoparticles fabricated at 0.57 mg/mL polymer concentration, 1:1 molar reactive site ratio, and three hours reaction time were used to investigate the possibility of colloidal gel fabrication and the effect of nanoparticles on the properties of HA suspensions.

### Example 2

### Colloidal Gels Composed of High Concentrations of HA Nanoparticles

Several characterization methods were employed to evaluate physical, mechanical, and rheological properties of colloidal gels composed of high concentrations of HA nanoparticles.

Materials

Hyaluronic acid and chondroitin sulfate nanoparticles synthesized as discussed above at 0.57 mg/mL polymer concentration, 1:1 molar reactive site ratio, and three hours reaction time were used to make colloidal gel systems. Nanoparticles made from 17 kDa HA, 1500 kDa HA, and ˜30 kDa CS were selected to investigate the effect of HA molecular weight and type of GAG on the potency of colloidal gel formation. HA (17 kDa and 1500 kDa) and CS (˜30 kDa) polymers were used to make controls. 1-Ethyl-3-[3-dimethylaminopropyl]carbodiimide hydrochloride (EDC) was purchased from Thermo Scientific (Rockford, Ill.). Adipic acid dihydrazide was purchased from Sigma-Aldrich (St. Louis, Mo.).

Methods

Fabrication of Colloidal Gel

Colloidal systems were formed by simply mixing nanoparticles with deionized water at different concentrations. All experiments were performed in triplicate. First, dried nanoparticles (HA or CS) were removed from a freezer and equilibrated at room temperature. Nanoparticles were weighted and transferred to different microtubes at concentrations from 1.4% w/v up to 60% w/v in deionized water. The mixture was transferred to cylindrical molds (5 mm diameter and ˜1.8 mm height) and left for at least five hours. Polymers of HA (17 kDa and 1500 kDa) and CS (˜30 kDa) were used as negative controls at similar concentrations to investigate the possible formation of physically entangled gels in polymer solutions. Positive controls were also formed by adding ECD and adipic acid dihydrazide (with similar molarity used in nanoparticle fabrication) to 17 kDa HA polymer solutions at 15%, 30%, and 45% w/v. Positive controls were also used to investigate the possibility of gel formation with carbodiimide chemistry at similar colloidal gel concentrations.

Characterization of Colloidal Gels Via FTIR

A Spectrum 100 FTIR Spectrometer (PerkinElmer, Inc., Massachusetts) was employed to confirm that no chemical side reaction took place during colloidal gel fabrication. The FTIR spectra of the starting polymer, nanoparticles, and colloidal gel were compared.

Swelling Experiment

To investigate the swelling behavior and stability of colloidal gels, a swelling experiment was carried out as previously described (DeKosky et al., 2010; Zawko et al., 2009; Segura et al., 2005). All experiments were performed in triplicate. Briefly, colloidal gels at 15%, 30%, and, 45% w/v nanoparticle concentrations made from 17 kDa HA nanoparticles were placed into an excess of water or 0.1 M PBS for at least 6 hours. The equilibrated colloidal gel samples were weighed and placed into a desiccator chamber. After 48 hours, the dried colloidal gels were weighed again and the swelling ratio of colloidal gels was calculated according to Equation (1) where Ws (mg) is the swollen weight and W_{D} (mg) is the dry weigh of colloidal gels:

$$Swelling ratio = \frac{( {W}_{S} - {W}_{D} )}{{W}_{D}} \times 100 \qquad Eq . ( 1 )$$

Compression Testing and Mechanical Analysis

Compression testing was performed to investigate the mechanical properties of samples after fabrication, after swelling in water, and after swelling in 0.1 M PBS. Colloidal gels made from 17 kDa HA nanoparticles at 15%, 30%, and 45% w/v were used in this experiment. First, samples were placed on a glass slide and the dimensions of the cylindrical colloidal gels were determined with calipers using a microscope (Nikon TS100F). Since the dimensions of samples changed after swelling in water and 0.1 M PBS, samples were initially punched to a diameter of approximately 5 mm. Then, a confined uniaxial compression test using a RSA-III dynamic mechanical analyzer (TA Instruments, Delaware) was performed. Sample height was directly measured from the instrument. Mineral oil was used to lubricate both compression plates to reduce any sample-plate adhesion and prevent sample drying during the test. Compression testing was performed at a rate of 0.005 mm/s to obtain a stress-strain curve. The results were then analyzed according to the neo-Hookean model for an ideal elastomer in which the slop of the stress-strain fraction curve (λ−1/λ², where λ=L/L₀) yields a straight line with a slope equal to the shear modulus (DeKosky et al., 2010). Young's modulus, the slope of the stress-strain curve, was also estimated from the liner portion of the stress-strain fraction curve. If the initial region was not a straight line, that region was not considered in calculations. Toughness of samples, the absorbed energy, was finally calculated by numerical integration of the stress-strain curve (DeKosky et al., 2010). All experiments were performed in triplicate.

Effect of pH and Ionic Strength on Mechanical Properties of HA Colloidal Gel

Studies have shown that pH and ionic strength are two important factors that must be considered for injected formulation. Moreover, the influence of these factors on HA structure and hydrodynamic radius is understood. To investigate the effect of pH and ionic strength on the formation of HA colloidal gels and their mechanical properties, phosphate buffer (phosphate buffer concentration=10 mM) was made at three different pH values (pH=6, 7.4, and 8). This range covers the reported local pH tolerance for subcutaneous injection (Fransson et al, 1996; Gatej et al., 2005; Dornhofer et al., 1998). Sodium phosphate was chosen as buffer at pH=6 since other buffers such as citrate are known to be painful for injection. Usually, an isotonic solution containing 150 mM salt was reported for injection. In order to explore an ionic strength range similar to isotonic solution, three different ionic strengths (100 mM, 150 mM, and 200 mM) were selected for the buffer formulations. The buffers were prepared by mixing sodium phosphate salt and sodium chloride salt in deionized water at the desired pH and ionic strength.

Viscoelasticity of HA Colloidal Gel after Fabrication

In order to investigate the viscoelasticity of HA colloidal gels, dynamic oscillatory rheological measurements were performed using an AR-G2 rheometer (TA Instruments, Delaware) equipped with a 20, 20 mm diameter cone-plate geometry at a controlled temperature of 25° C. HA colloidal gel samples were prepared by mixing nanoparticles (made from 17 kDa HA) at different concentrations (5%, 15%, 30%, 45% w/v) in deionized water. 17 kDa HA polymer was also dissolved in deionized water at 45% w/v concentration as negative control. First, strain sweep experiments were performed to determine the limit of viscoelasticity, where the rheological properties are strain dependent. Then, the viscoelastic properties of colloidal gels were evaluated using a frequency sweep from 0.1 to 10 Hz at 1% strain, which was in the linear viscoelastic region. This range includes the physiological frequencies of the knee, ranging from 0.5 Hz (walk) to 3 Hz (running). All experiments were performed in triplicate.

In the oscillatory experiment, a sinusoidal shear strain was applied to the samples accordingly to:

γ=γ▪sin(ωt) Eq. (2)

where γ▪is the shear strain amplitude, ω is the oscillation frequency and t is the time. The mechanical response, expressed as the shear stress (τ) of viscoelastic materials, ranges from an ideal pure viscoelastic solid (follows Hooke's law) and an ideal pure viscous fluid (follows Newton's law) (Barbucci et al., 2002). As a result, the mechanical response is out of phase with respect to the imposed deformation as stated by:

τ=G′(ω)γ▪sin(ωt)+G″(ω)γ▪cos(ωt) Eq. (3)

where G′(ω) is the shear storage modulus giving information about elasticity or the energy stored in the material during deformation and G″(ω) is the shear loss modulus which describes the viscous characteristic or the energy scattered as heat (Barbucci et al., 2002).

The combined viscous and elastic behavior is defined by the absolute value of complex shear modulus G⁻ (Barbucci et al., 2002):

G⁻(ω)=\sqrt{G′²+G″²} Eq. (4)

The ratio between the viscous modulus and the elastic modulus is defined by the loss tangent (tan delta):

$$tan δ = \frac{{G}^{′}}{{G}^{″}} \qquad Eq . ( 5 )$$

The loss tangent is the ratio of the energy lost to the energy stored in the cyclic deformation.

Viscosity of HA Colloidal Gel

The viscosity of the different formulations was determined using an AR-G2 rheometer (TA Instruments, Delaware) equipped with a 20, 20 mm diameter cone-plate at 25° C. HA nanoparticles (made from 17 kDa HA) were mixed with deionized water at different concentrations (5%, 15%, 30%, 45% w/v). Shear stress and viscosity of the samples were measured over a shear rate sweep of 0.01-100 s⁻¹. 17 kDa HA polymer was also dissolved in deionized water at 45% w/v concentration as a negative control. All experiments were performed in triplicate.

HA Colloidal Gel Recoverability: Mechanical Dynamics and Recoverability

To characterize recoverability and the dynamic behavior of HA colloidal gels, compression testing was performed as described above. Two sets of samples (after fabrication and swollen in 0.1 M PBS) were prepared for this experiment. After fabrication of colloidal gels at different nanoparticle concentrations (15%, 30%, and 45% w/v), one set of samples was swollen in 0.1 M PBS solution for at least 24 hours. Each sample was punched to a diameter of approximately 5 mm. After loading each sample, five preconditioning compression cycles at 6% strain were applied to initiate the recoverability experiment. Then, a compression at 30% strain (0.005 mm/s) was applied. After squeezing the sample, compression force was removed and the sample was allowed to relax for five minutes. Meanwhile, the sample was hydrated with deionized water to prevent dryness. Finally, another compression at 30% strain (0.001 mm/s) was applied. The mechanical recoverability and dynamic property of samples were identified by comparing the recovered height of the samples after the second compression to the height of the samples before the first compression. Normal stress-gap curves were generated and used to identify the recoverability. All experiments were performed in triplicate.

Rheological Recoverability

To evaluate the rheological recoverability of colloidal gels, the viscosity was measured as described above. Samples at 15% w/v nanoparticle concentration in deionized water were prepared for this experiment. Then, the colloidal gel was loaded under the rheometer and three cycles of shear rate sweeps (0.01-100 s⁻¹) with a one minute delay between each cycle were applied to the samples. The changes in shear stress and viscosity over shear rate sweep after each run was evaluated. All experiments were performed in triplicate.

Physical Recoverability

To evaluate the physical recoverability of the colloidal gel, samples at 30% w/v nanoparticle concentration in deionized water were prepared. The samples were swollen in water for at least 24 hours. Then, the swollen samples were completely crushed and dried in a desiccator for at least 48 hours. Finally, dried samples were weighed and again the colloidal gel samples at 30% w/v were remade. The physical recoverability of the samples was evaluated by visual observation. All experiments were performed in triplicate.

Statistical Analysis

Statistical analysis was performed by one-way analysis of variance (ANOVA). To evaluate the significance of differences, Newman-Keuls was used as a post-hoc test. A value of p<0.05 was accepted as significant in all cases.

Results

Fabrication of Colloidal Gel

Colloidal gels were formed by mixing nanoparticles with deionized water at different concentrations. Samples were made from 17 kDa and 1500 kDa HA (FIG. 8). These nanoparticles were assumed to have different structural properties. 17 kDa HA nanoparticles were suspected to have a more ‘hairy’ structure with dangling chains compared to 1500 kDa HA nanoparticles. Colloidal gels with a stable 3-D structure formed at 15%, 30%, and 45% w/v when nanoparticles made from 17 kDa HA where used. Colloidal gels could also hold their structure upon swelling in deionized water. At 5% w/v nanoparticle concentration, the mixture could not form a stable 3-D structure; however, a viscous suspension of HA nanoparticles was obtained (FIG. 8, B). That might be because of the association or physical entanglement between dangling chains of nanoparticles. On the other hand, 17 kDa HA polymer solutions at 5%, 15%, 30%, and 45% were liquid and a 3-D network was not evident (FIG. 8, A). Even positive controls using EDC and adipic acid dihydrazide with hyaluronic acid resulted in no gel formation at similar concentrations. Positive controls were made by activating carboxyl groups of 17 kDa HA via EDC and trying to crosslink them via adipic acid dihydrazide in deionized water (but without acetone in this case) at similar concentrations to colloidal gel formulations (FIG. 8, C).

Mixing nanoparticles fabricated from 1500 kDa HA at 1.4%, 5%, 10%, 15%, 30%, and 45% w/v concentrations did not form any stable colloidal gel network (FIG. 8, E). At lower concentrations (1.4%, 5%, 10%, and 15% w/v), formation of a low viscosity nanoparticle suspension was observed; however, at 30% and 45% w/v nanoparticle concentrations, paste-like materials were obtained (FIG. 8, E and F). Mixing 1500 kDa HA polymer with deionized water at 1.4% w/v resulted in formation of a viscous polymer solution however at 5%, 10%, and 15% w/v polymer concentrations, 3-D gels formed (FIG. 8, D). These gels, however, could be dissolved while swelling in deionized water.

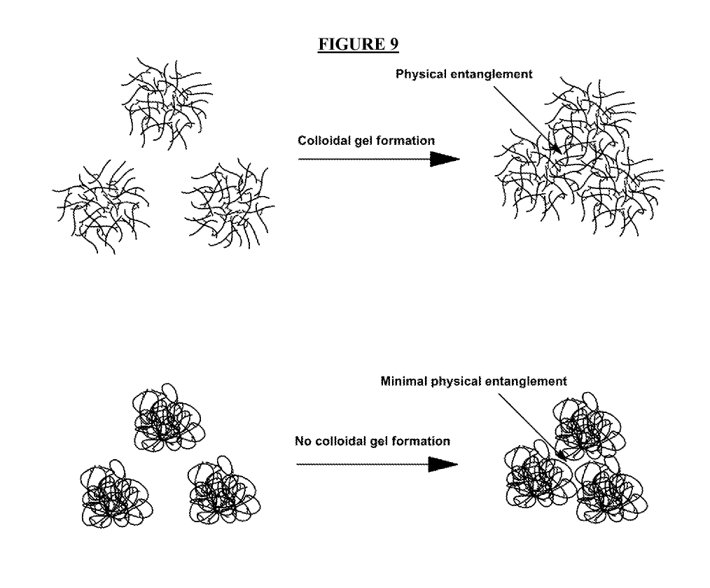

Results showed that at 15%, 30%, and 45% w/v nanoparticle concentrations (17 kDa HA nanoparticles), colloidal gels with stable 3-D structure were formed. In contrast, paste-like materials were formed in the samples made from 1500 kDa HA nanoparticles at similar concentrations. Formation of stable colloidal gels in the samples made from 17 kDa HA nanoparticles may be due to association or physical entanglement of the dangling chains available on the surface of 17 kDa HA nanoparticles. On the other hand, the absence of these dangling chains on the nanoparticles made from 1500 kDa HA negated physical entanglement and yielded weak paste-like materials (FIG. 9).

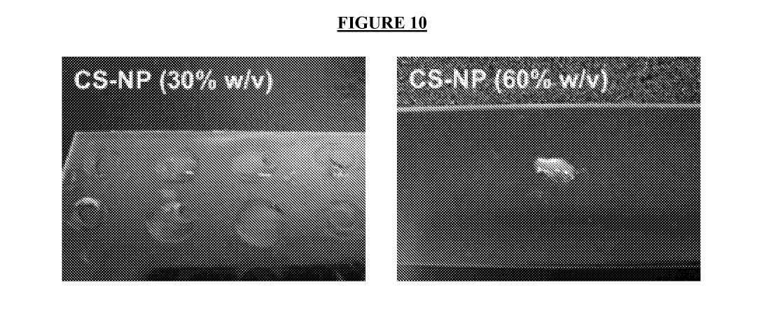

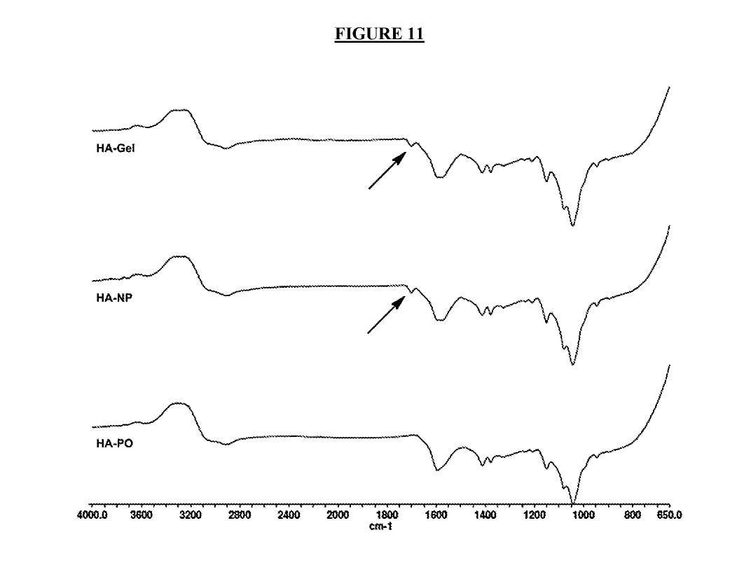

Chondroitin sulfate (CS) (˜30 kDa) nanoparticles were also tested; however, a stable 3-D structure network did not form even at high concentration of CS nanoparticles (60% w/v). Mixing CS nanoparticles with deionized water only formed colloidal suspensions. This might be due to the absence of physical properties such as entanglement and self-association of CS polymer chains compared to hyaluronic acid (FIG. 10).

Characterization of Colloidal Gels Via FTIR

FTIR spectra of HA polymer, HA nanoparticles, and HA colloidal gel at 30% w/v nanoparticle concentration (17 kDa HA) showed that no chemical side reaction occurred during the colloidal gel fabrication process (FIG. 11).

Swelling Experiment

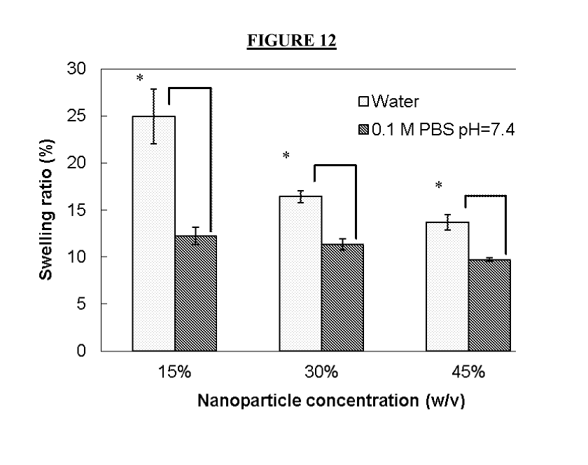

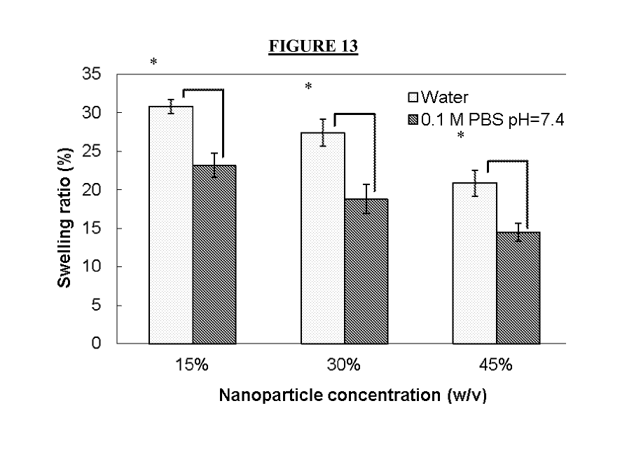

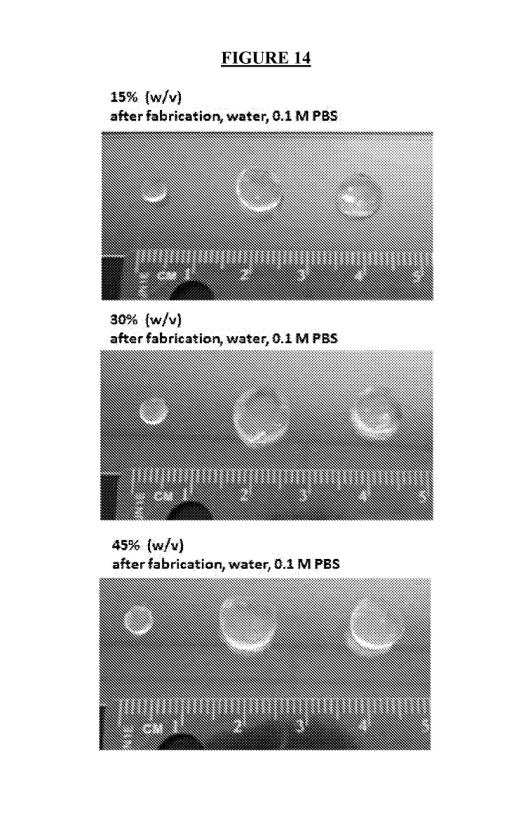

The swelling ratio of HA colloidal gels at different nanoparticle (17 kDa HA) concentrations (15%, 30%, and 45% w/v) were measured at 6 and 24 hours (FIG. 12 and FIG. 13). The swelling ratio of samples swollen in deionized water was greater than the swelling ratio of samples swollen in 0.1 M PBS. The presence of salt in 0.1 M PBS solution may keep the HA structure more coiled with a lower hydrodynamic radius resulting in less swelling of nanoparticles. Increasing nanoparticle concentration decreased the swelling ratio for the samples swollen in both water and 0.1 M PBS. More association or physical entanglement between nanoparticles was expected at higher concentrations. Finally, HA colloidal gels swollen in water and 0.1 M PBS could hold their stable 3-D structures (FIG. 14).

Compression Testing and Mechanical Analysis

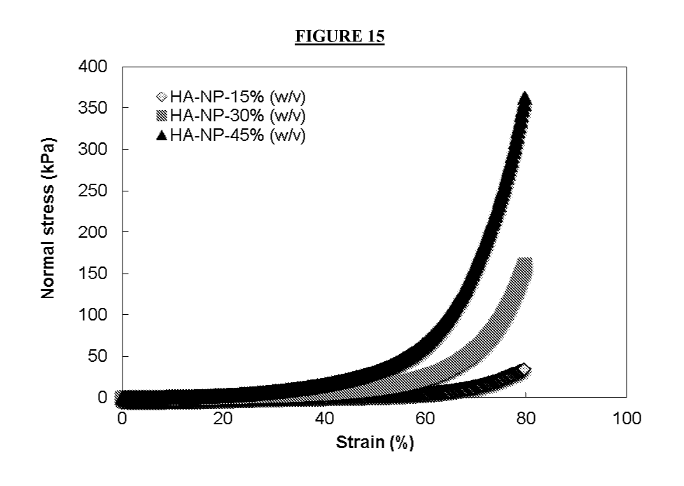

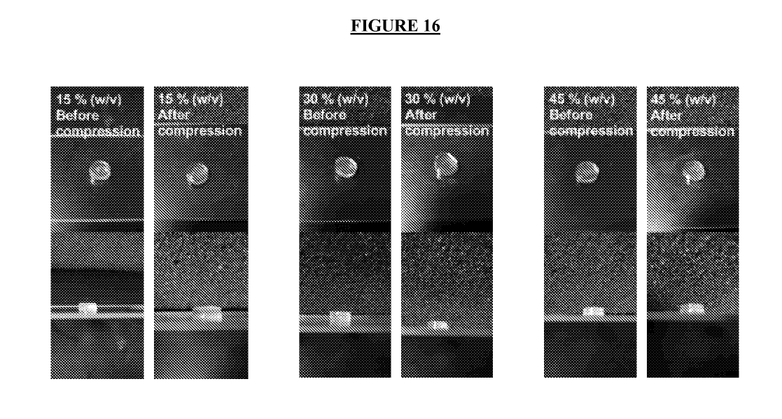

Uniaxial compression testing was performed on colloidal gels at 15%, 30%, and 45% w/v nanoparticle concentrations (17 kDa HA) after fabrication, and after swelling in deionized water or 0.1 M PBS. Stress-strain curves collected for colloidal gels after fabrication suggested that the colloidal gels behave as soft elastic materials without a failure point (FIG. 15). Samples did not even break during compression testing up to 80% strain. After removing the force, samples could recover their initial shape over time (˜5 minutes) before and after compression testing up to 80% strain (FIG. 16).

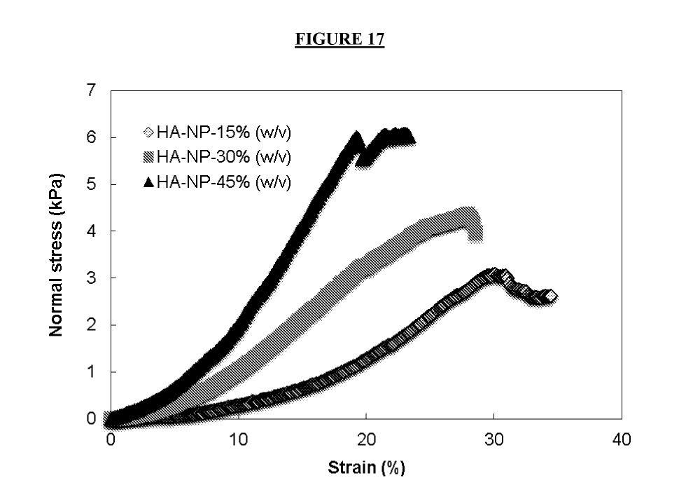

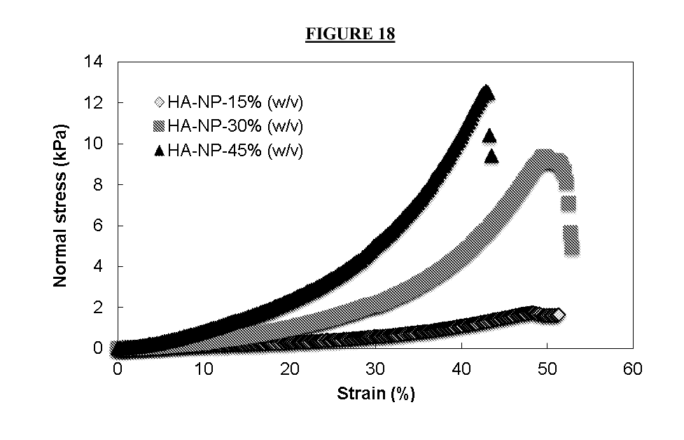

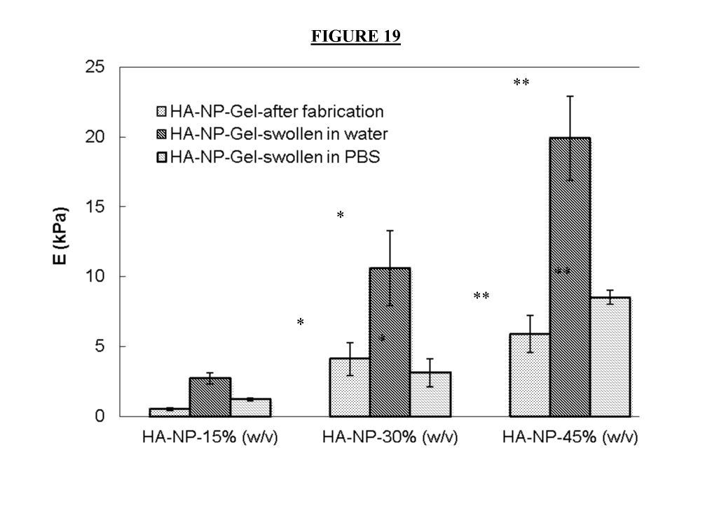

In addition, stress-strain curves were collected for the samples swollen for 24 hours in deionized water (FIG. 17). Samples at different nanoparticle concentrations broke with different stress and strain values (Table 3-1). Increasing nanoparticle concentration increased the slope of the curves indicating stiffer materials (FIG. 17 and FIG. 19). Similar behavior was also observed for the samples swollen in 0.1 M PBS. Swollen samples failed at different stress strain values depending on the nanoparticle concentration (Table 3-1). Increasing nanoparticle concentration increased the stiffness (the slope of stress-strain curves) of the samples (FIG. 18).

Young's modulus (E) is defined as the initial slope of stress-strain curves indicating the stiffness of material. Young's modulus of colloidal gels was determined at different conditions (FIG. 19). Increasing nanoparticle concentration increased Young's modulus. This increase was greater for the samples swollen in deionized water. Moreover, samples swollen in deionized water had the highest Young's modulus. Perhaps due to the decrease in chain entanglement in 0.1 M PBS due to ionic strength.

In addition, the Young's modulus of colloidal gel samples at 15%, 30% and 45% w/v were statistically different (FIG. 19). Increasing nanoparticle concentration significantly increased the Young's modulus of the samples swollen in deionized water and 0.1 M PBS. Based on these results, Young's modulus is a concentration dependent factor of HA colloidal gels and that may be influenced by the environment of the samples (FIG. 19).

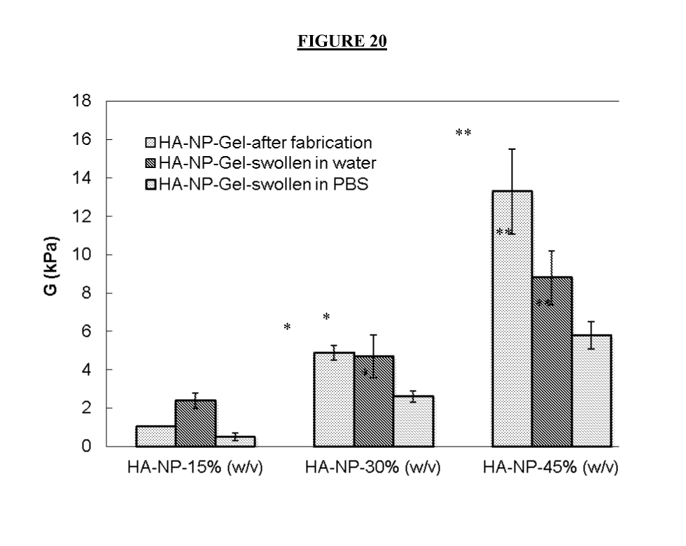

Based on the neo-Hookean model, the initial slope of the stress-stain curve (where the curve is linier) is identified as the shear modulus (G), the rigidity of material (DeKosky et al., 2010). The shear modulus of colloidal gels was determined at different conditions (FIG. 20). Increasing nanoparticle concentration increased the shear modulus for all conditions; however, this increase was more pronounced in the samples after fabrication. Similar to Young's modulus, nanoparticle concentration and salt content influenced the shear modulus.

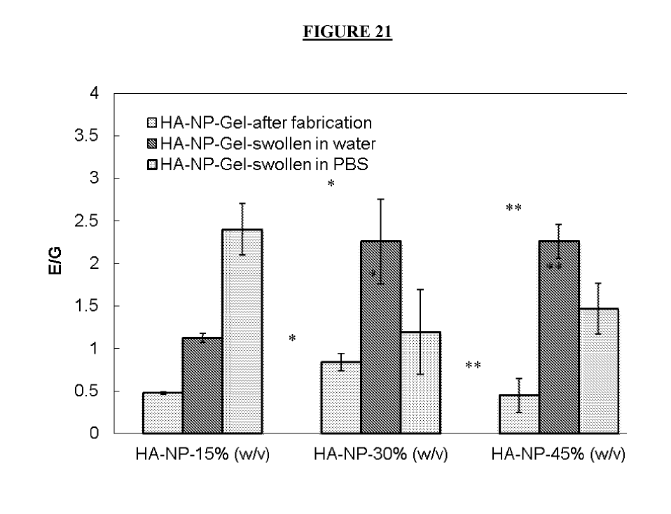

For incompressible materials such as hydrogels, E was reported to be approximately equal to 3G (DeKosky et al., 2010). E/G values identify the relationship between the stiffness and rigidity of the material and E/G was calculated for colloidal gels at different conditions (FIG. 21). Obviously, colloidal gels did not behave as an incompressible material. The data suggested that Young's modulus is less than 3G. Increasing nanoparticle concentration from 15% to 30% w/v increased E/G for the samples swollen in deionized water. In contrast, increasing nanoparticle concentration decreased E/G values for the samples swollen in 0.1 M PBS at similar concentrations. The influence of nanoparticle concentration on E/G did not follow a similar pattern for all sample conditions.

Compressive failure properties were determined for samples swollen in deionized water or 0.1 M PBS (Table 3-1). Strain failure of samples swollen in water was significantly lower than the strain failure of samples swollen in 0.1 M PBS at all nanoparticle concentrations. Increasing nanoparticle concentration, increased stress failure and toughness for samples swollen in deionized water or in 0.1 M PBS. The toughness of samples swollen in deionized water was statistically different from the toughness of samples swollen in 0.1 M PBS at all concentrations (Table 3-1). It could be concluded that the increase of failure stress and toughness might be due to greater physical entanglement between nanoparticles at higher nanoparticle concentrations.

TABLE (3-1)
Compressive failure properties of colloidal gels swollen in deionized water
or 0.1M PBS. Data represent the mean ± SD (n = 3).

| | Compressive failure properties | | | | | |
|---|---|---|---|---|---|---|
| | Swollen in water | | | Swollen in 0.1M PBS | | |
| Gel | Strain | Stress | Toughness | Strain | Stress | Toughness |
| formulation | (%) | (kPa) | (kJ/m³) | (%) | (kPa) | (kJ/m³) |
| HA-NP 15% | 30 ± 2.1 | 3 ± 0.16 | 0.3 ± 0.032 | 53 ± 3.4 | 1 ± 0.7 | 0.5 ± 0.082 |
| (w/v) | | | | | | |
| HA-NP 30% | 26 ± 3.4 | 5 ± 1.6* | 0.6 ± 0.183 | 60 ± 1.5 | 10 ± 0.24 | 2.7 ± 0.365* |
| (w/v) | | | | | | |
| HA-NP 45% | 21 ± 1.4** | 6 ± 2.5** | 0.6 ± 0.234 | 48 ± 2.6 | 16 ± 4.6 | 3.3 ± 0.667** |
| (w/v) | | | | | | |

* and **p < 0.05 comparing to HA-NP 15% w/v.

Effect of pH and Ionic Strength on Mechanical Properties of HA Colloidal Gel

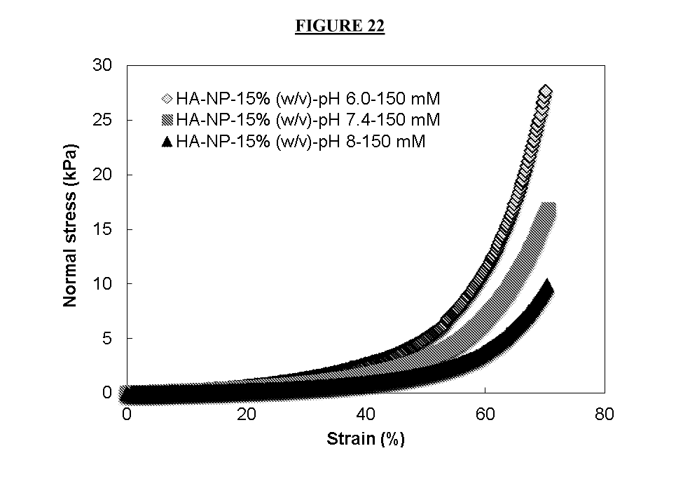

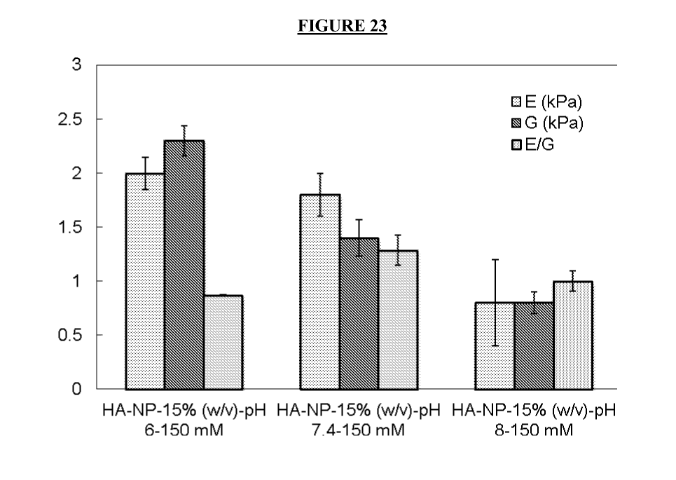

The stress-strain curves were determined for samples at 15% w/v nanoparticle concentration and different pH values (17 kDa HA) (FIG. 22). Increasing pH influenced the behavior of material as was observed from the calculated Young's modulus and shear modulus which showed that the material become softer at higher pH values. Increasing pH at 150 mM salt concentration decreased both Young's modulus and shear modulus; however, this decrease was not significant for all the samples. Increasing pH can reduce the hydrodynamic radius of HA. Therefore, the association or physical entanglement between nanoparticles may decrease at higher pH values resulting in formation of softer materials. The highest values for Young's modulus and shear modulus occurred at pH=6.0 (FIG. 23).

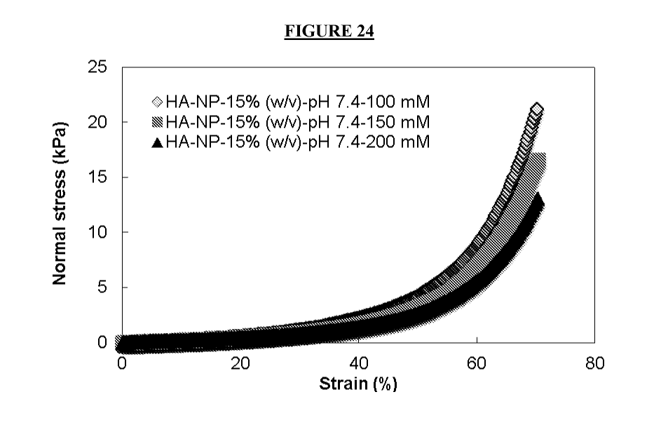

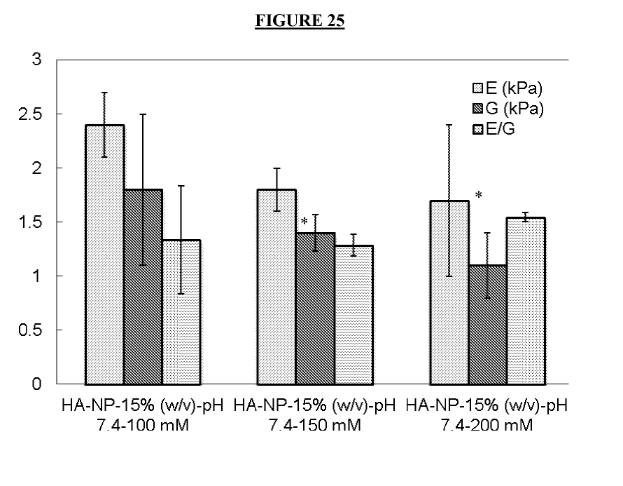

Changing the salt concentration at a constant pH of 7.4 also affected the mechanical properties of colloidal gels at 15% w/v nanoparticle concentration (17 kDa HA). Increasing salt concentration made colloidal gels softer (FIG. 24). Young's modulus and shear modulus appeared greater at lower salt concentrations; however, the results were not statistically significant (FIG. 25). The presence of salt in the formulations can influence the structure and reduce HA hydrodynamic radius. Therefore, this effect may also decrease physical entanglement between nanoparticles leading to a softer material (FIG. 25)

Viscoelasticity of HA Colloidal Gel after Fabrication

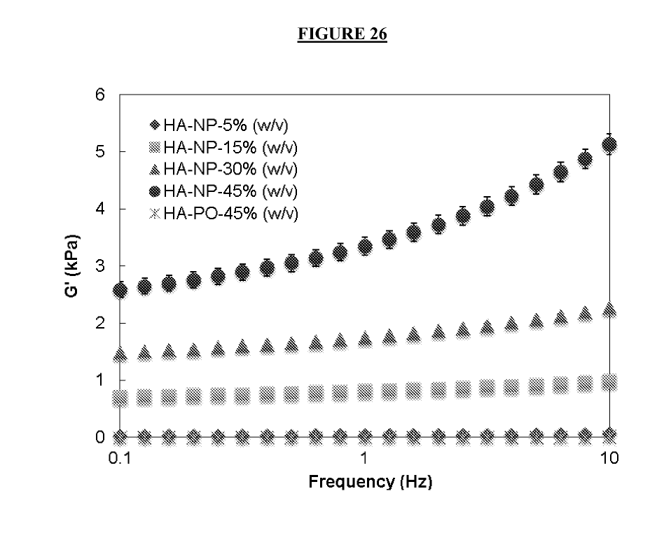

Viscoelastic properties were evaluated for HA colloidal gels using different nanoparticle concentrations. Nanoparticles made from 17 kDa HA were used to make colloidal gels at 5%, 15%, 30%, and 45% w/v. 17 kDa HA polymer at 45% w/v in deionized water was also used as a control. The changing storage modulus (G′) of colloidal gels was determined over a frequency range of 0.1-10 Hz (FIG. 26). Increasing nanoparticle concentration increased the storage modulus of colloidal gels. Samples at 5% w/v nanoparticle concentration had a similar storage modulus compared to the 45% polymer solution. The storage modulus of other colloidal samples was greater than the control.

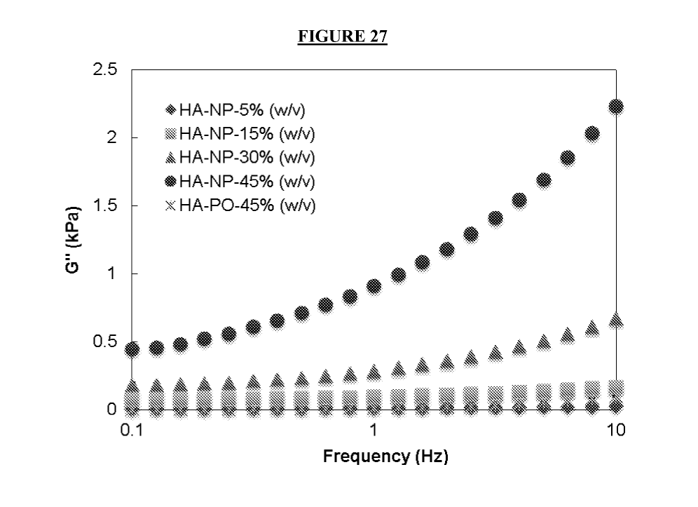

Increasing nanoparticle concentration also increased the loss modulus (G″) of colloidal gels (FIG. 27). Samples at 5% w/v nanoparticle concentration showed a similar loss modulus compared to the 45% polymer solution. The loss modulus of the other colloidal samples was greater than the control.

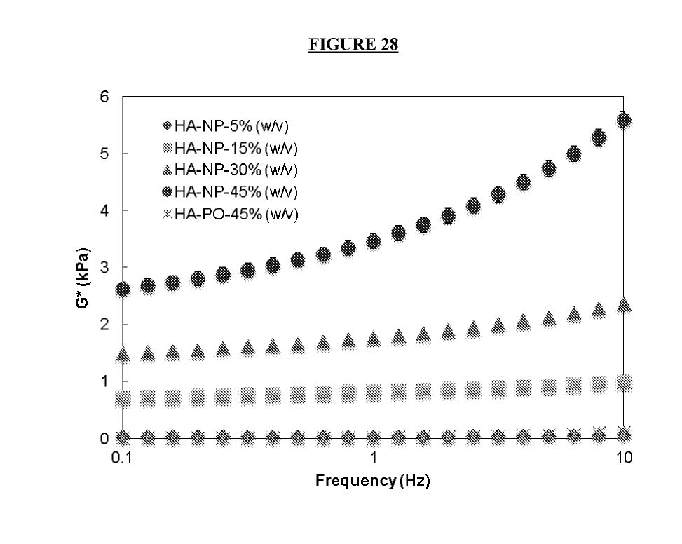

The calculated complex modulus (G*) showed behavior similar to the storage and loss moduli over frequency range (FIG. 28). Samples at 5% w/v nanoparticle concentration had a similar complex modulus compared to 45% polymer solution.

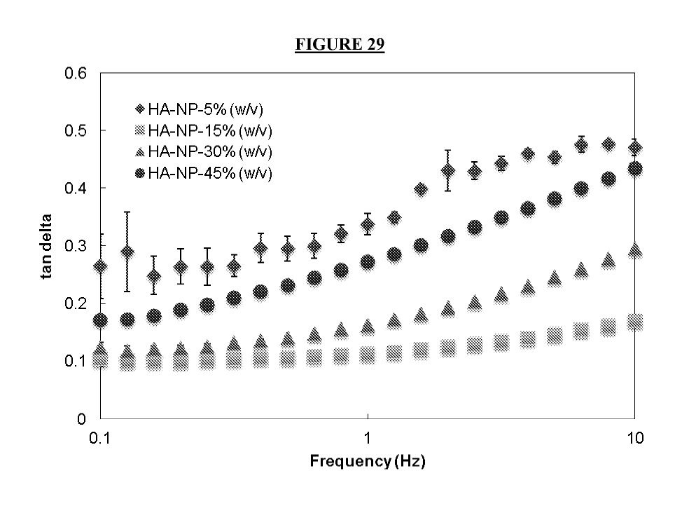

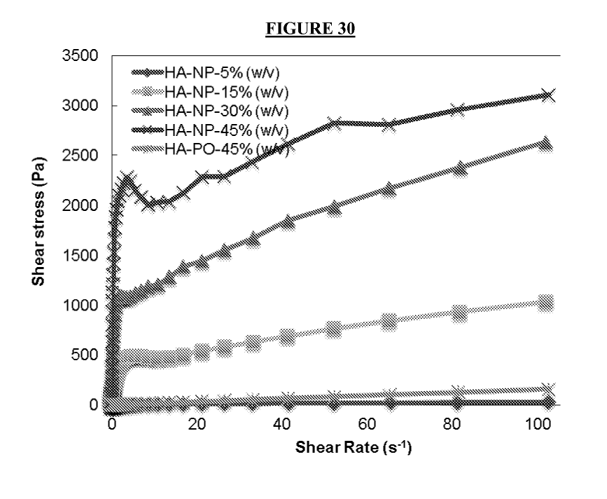

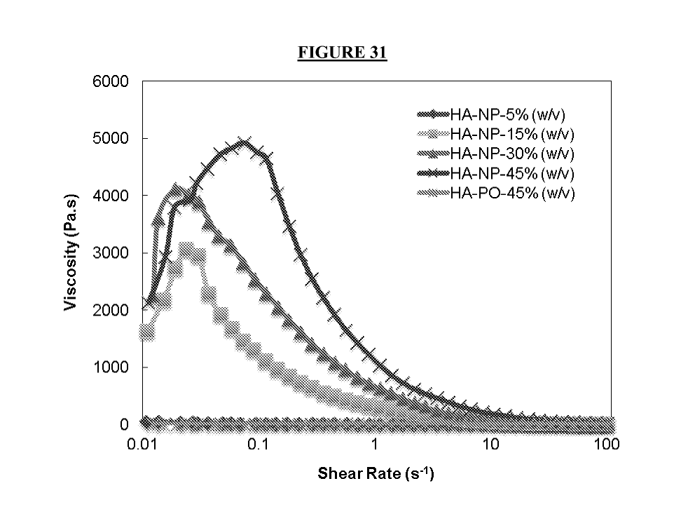

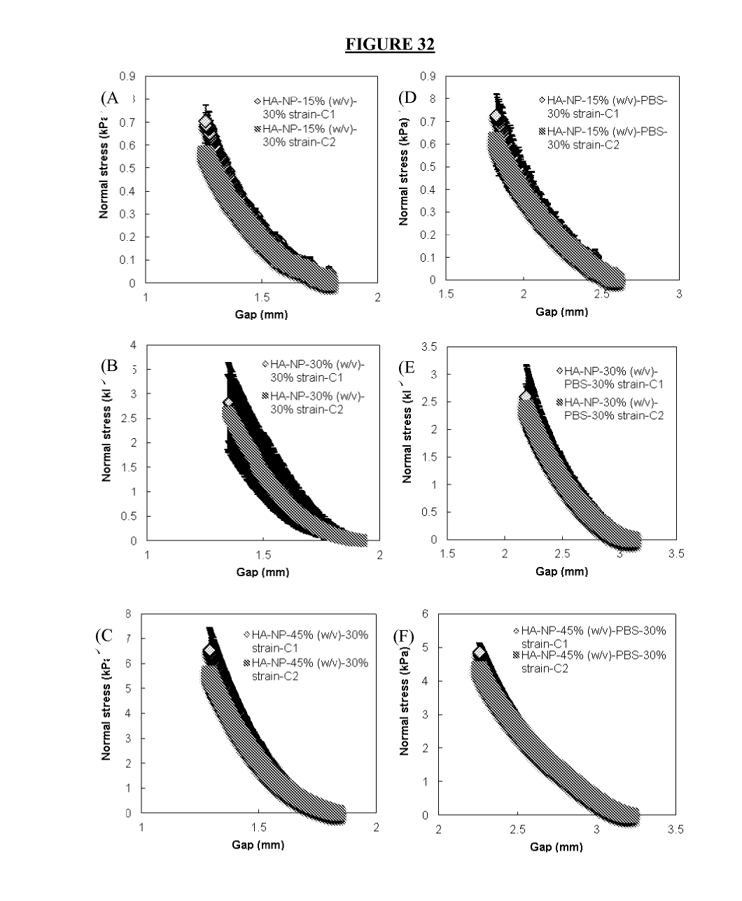

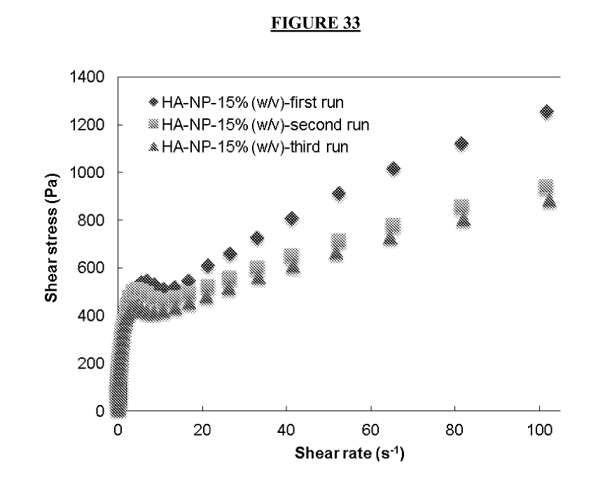

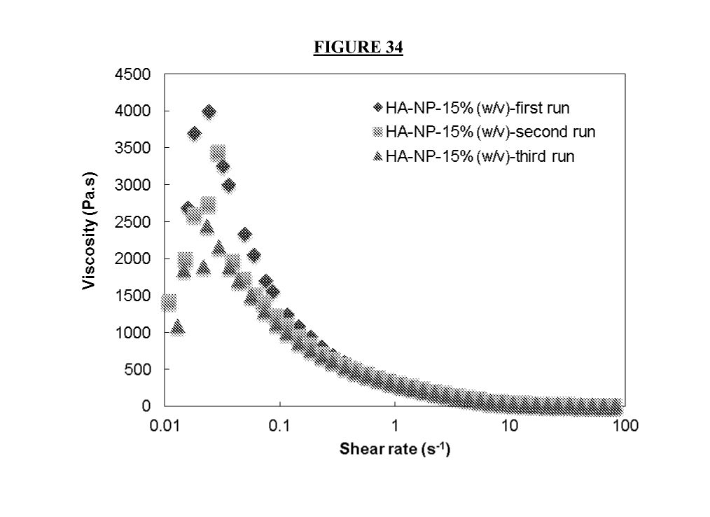

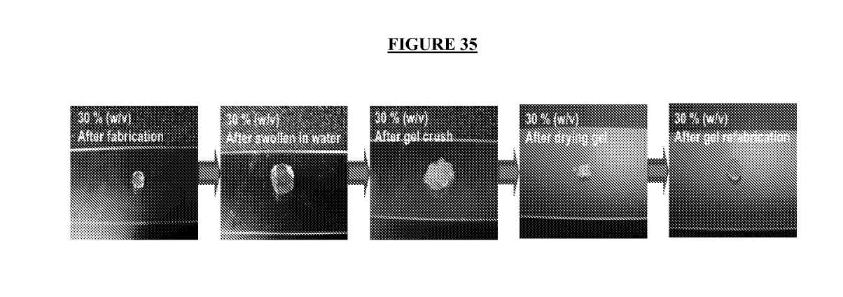

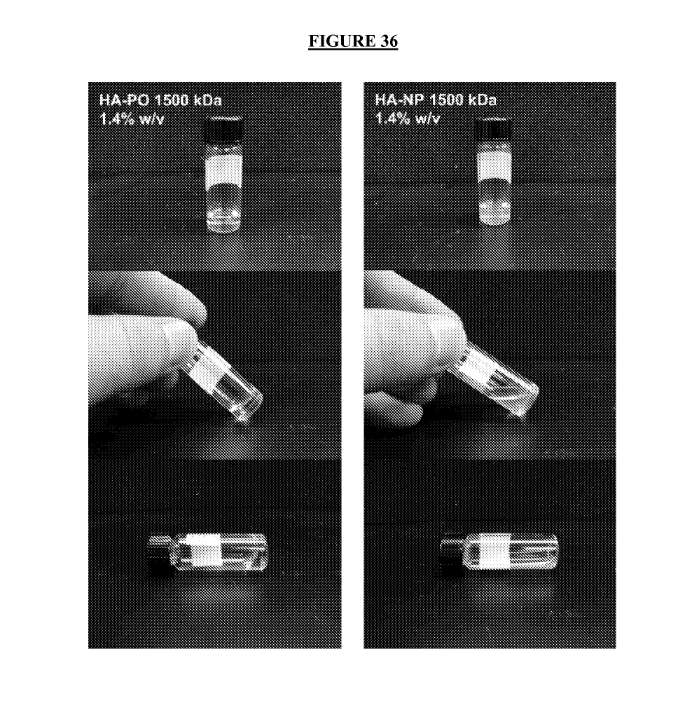

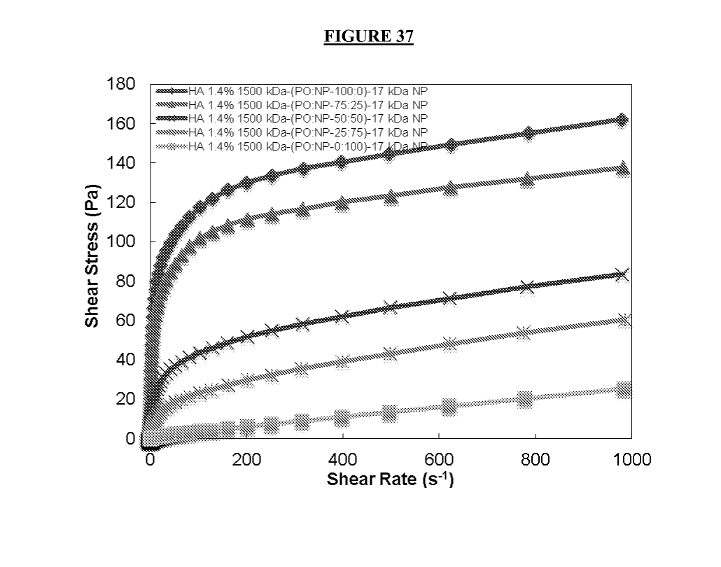

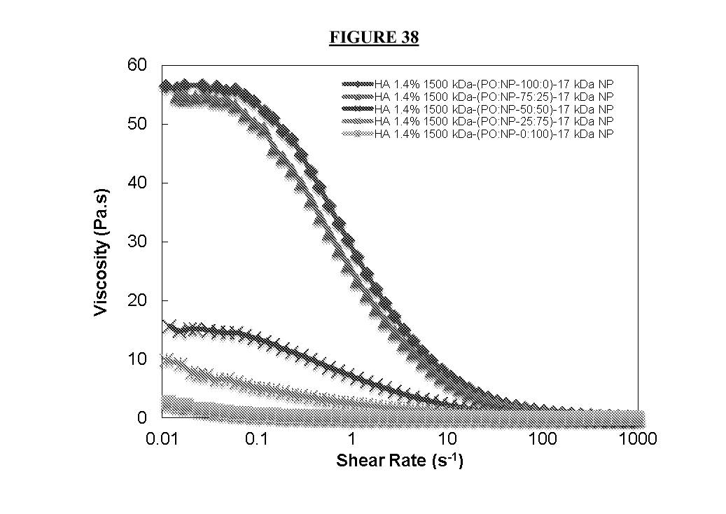

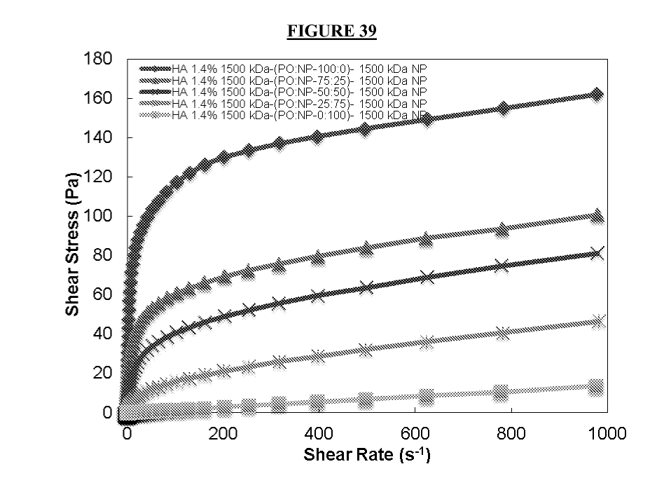

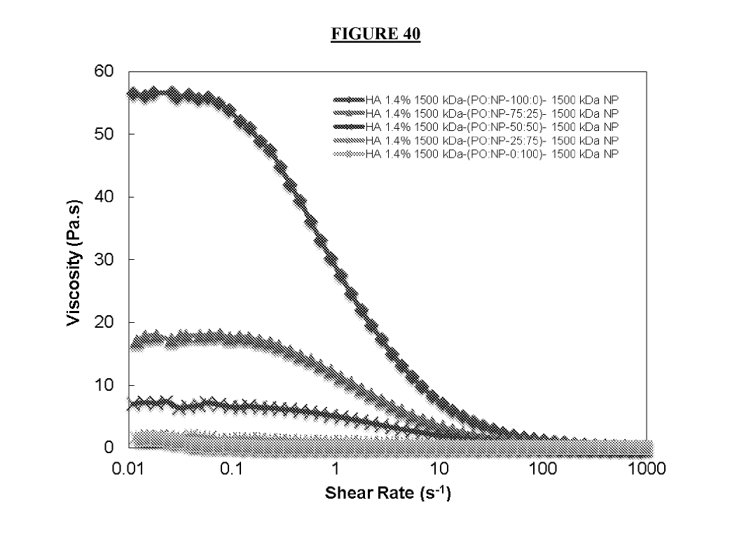

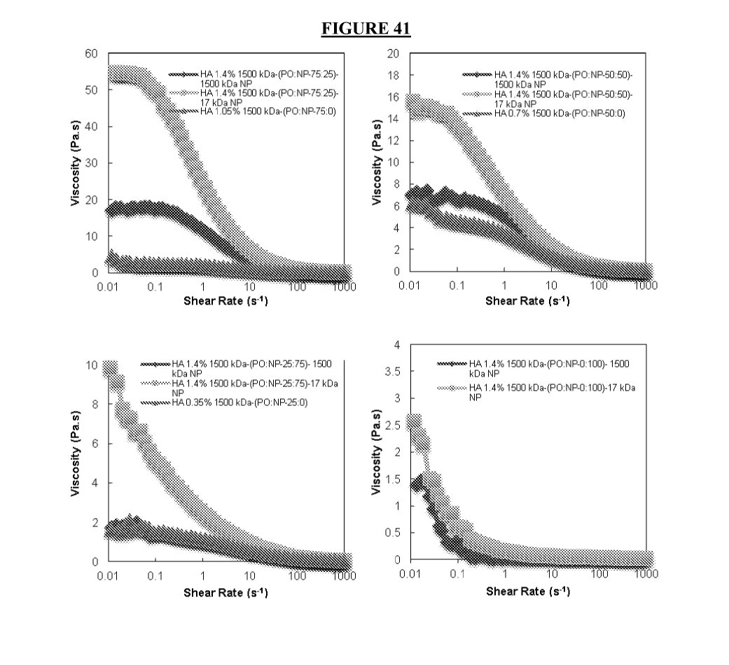

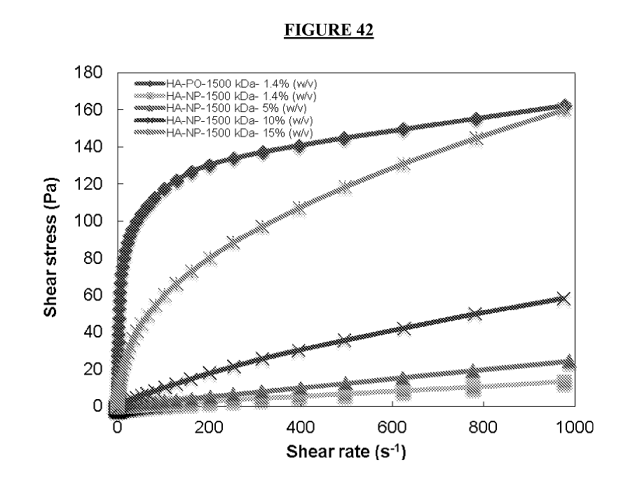

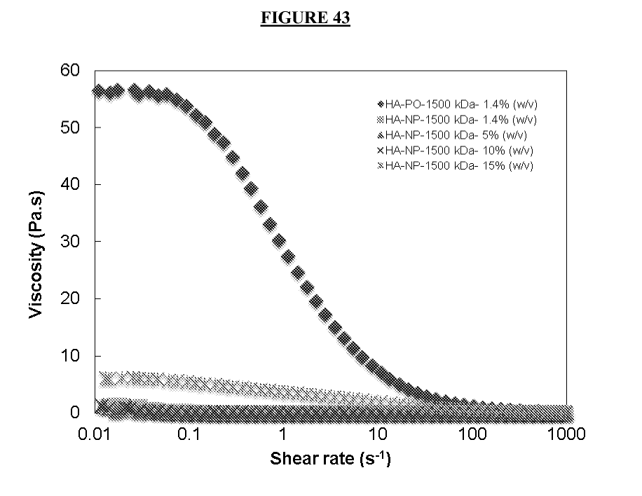

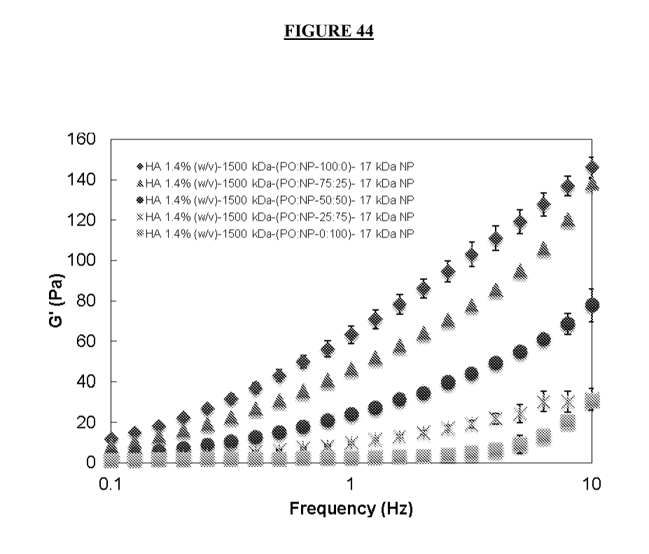

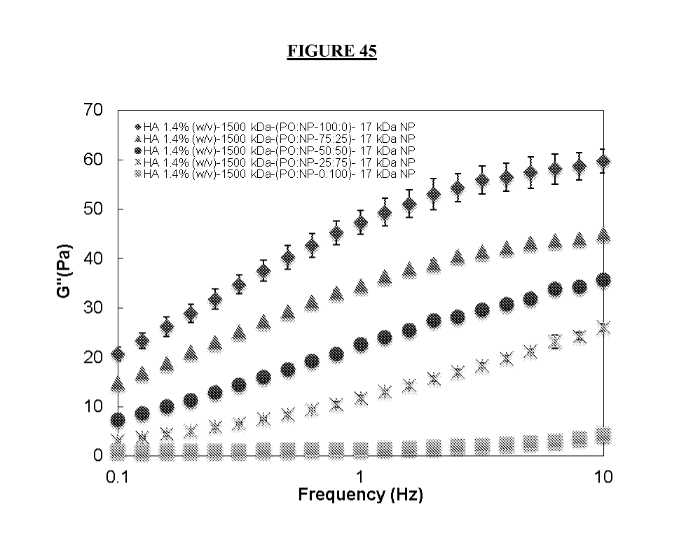

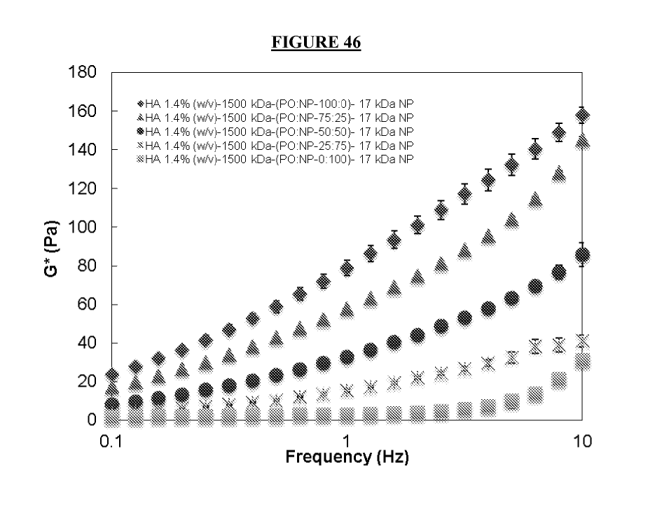

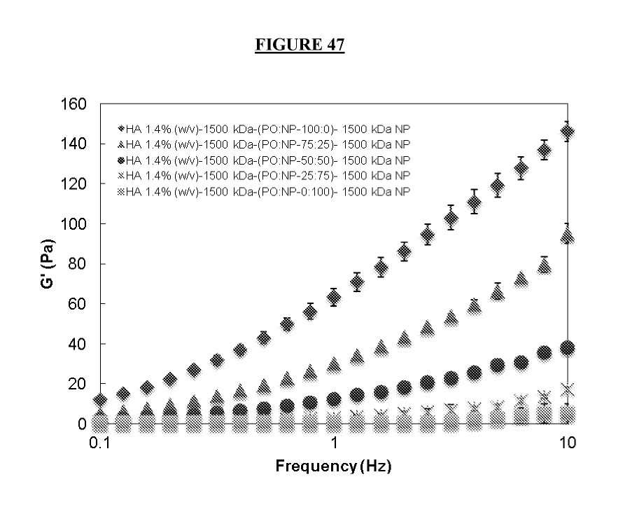

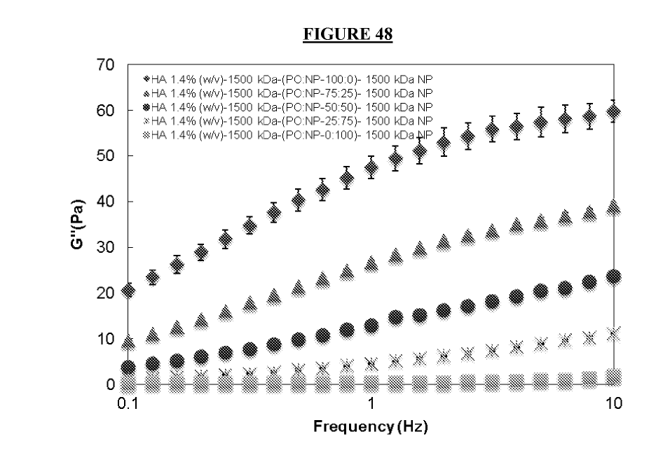

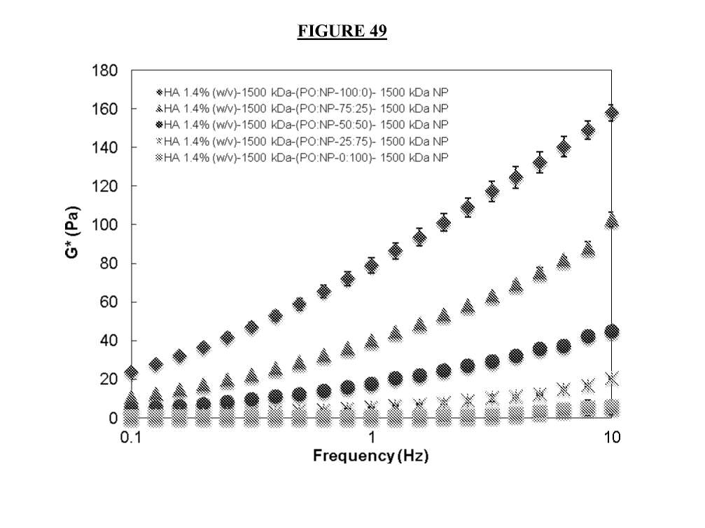

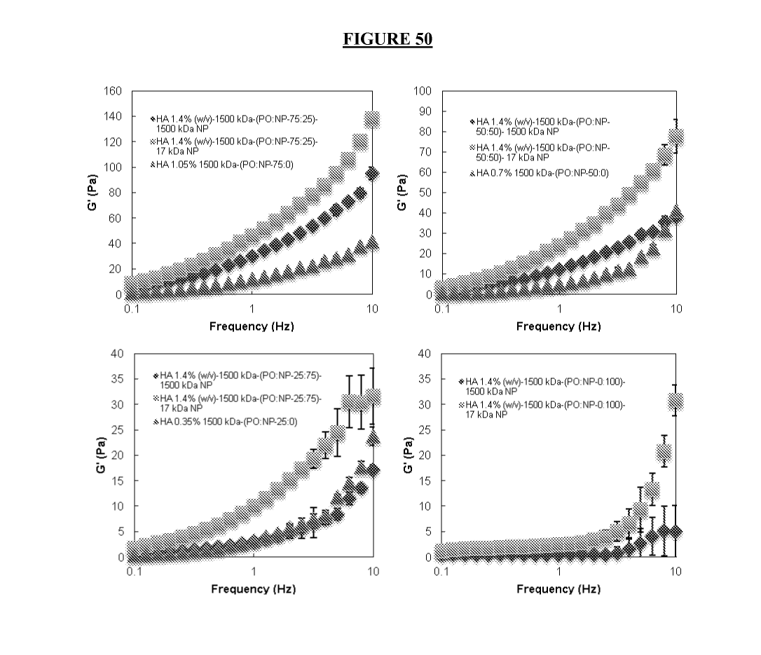

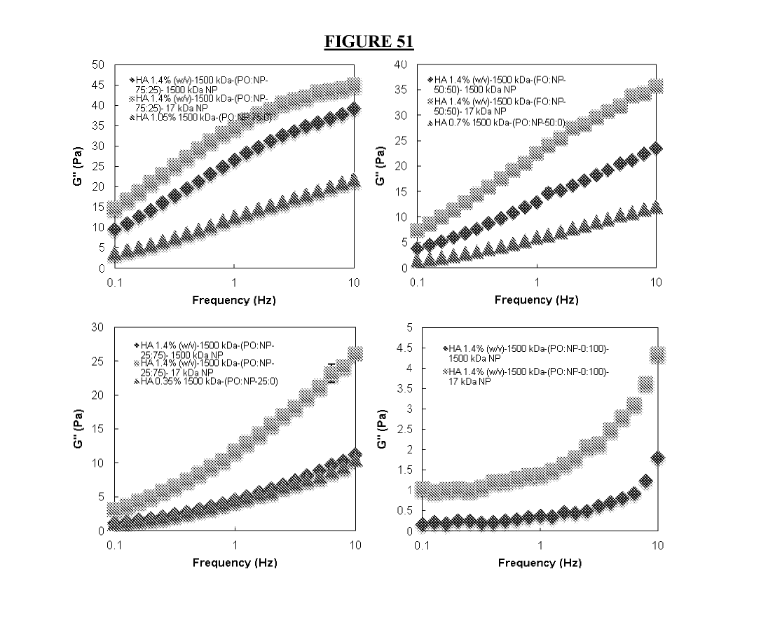

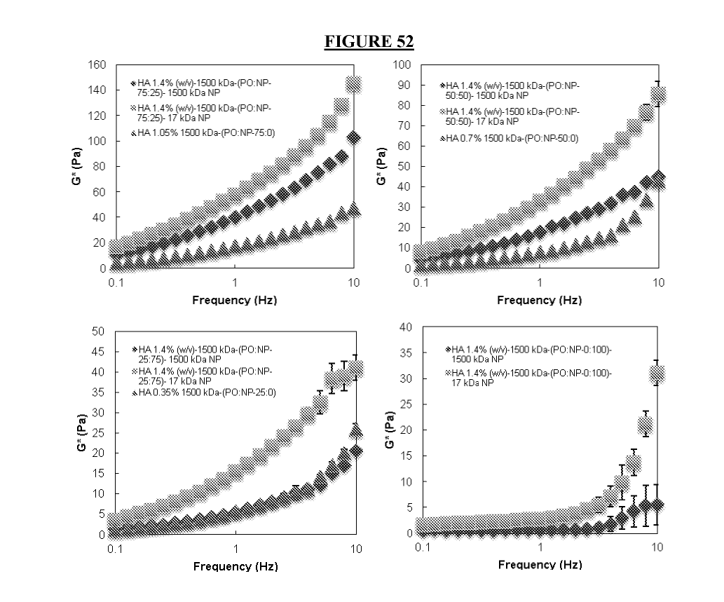

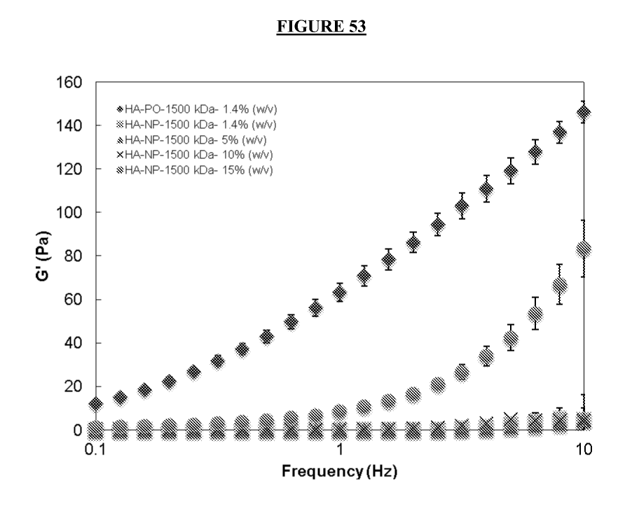

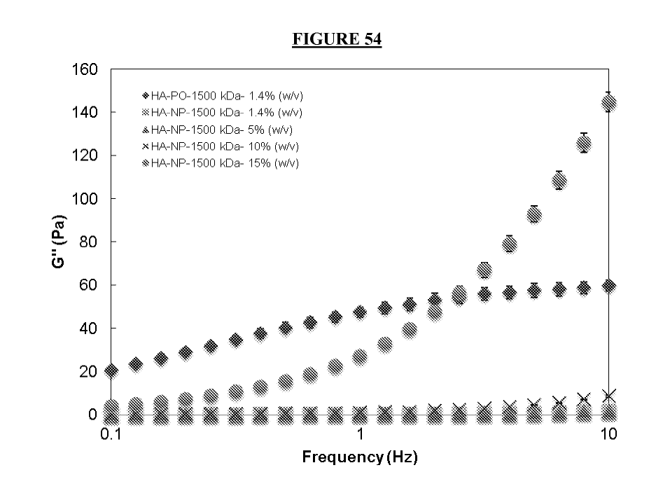

Excluding data for the sample at 5% w/v concentration, tan delta was also increased by increasing the concentration of nanoparticles (FIG. 29). Since a stable 3-D structure was not formed at 5% w/v, the calculated tan delta for this sample showed a different behavior compared to the rest of the samples.

Viscosity of HA Colloidal Gel

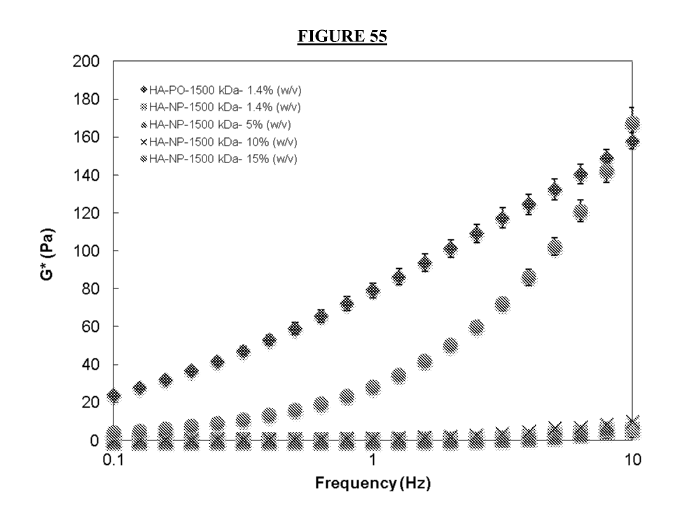

The viscosity of colloidal gels was measured at different nanoparticle concentrations in deionized water (FIG. 30). Changes in viscosity over shear rate sweep are also presented (FIG. 31). The 17 kDa HA polymer solution at 45% w/v concentration was used as a control to compare the behavior of colloidal gels with polymer solution.

Results showed that samples at 5% w/v behaved similar to the control since a colloidal gel network did not form at that concentration. By increasing nanoparticle concentration up to 15% w/v and higher, the behavior of the samples changed. A yield point was observed for the samples at 15%, 30%, and 45% w/v concentrations. In addition to that, increasing nanoparticle concentration increased shear stress and viscosity of the samples. At lower shear rates, the viscosity of samples increased to a maximum (yield point) and then dropped by increasing shear rate. This yield point correlated to the yield stress point on shear stress-shear rate curves. Increases in the viscosity and shear stress up to the yield point might be due to the interlocking of dangling chains available on the surface of nanoparticles (FIG. 31). After the yield point, nanoparticles may thus lowering the shear stress and viscosity at higher shear rates. The colloidal gels shear thinned after the yield point, which indicated a “pseudo-plastic” material (FIG. 31).

HA Colloidal Gel Recoverability: Mechanical Dynamics and Recoverability

Recoverability of colloidal gels at 15%, 30%, and 45% w/v nanoparticle concentrations (17 kDa HA) tested after fabrication and after swelling in 0.1 M PBS. Representative stress-gap curves were collected for all the tested formulations (FIG. 32). Results suggested that after the first cycle of compression/decompression, the height of the samples recovered back to the initial height. This might be due to the structural properties of the colloidal gel samples including chemical crosslinking in the nanoparticles, physical entanglement between nanoparticles, and the nature of HA itself. Mechanical dynamics and recoverability did not depend on nanoparticle concentration. Moreover, similar recoverability behavior was observed for both conditions (after fabrication and after swelling samples in 0.1 M PBS). Due to the failure of samples swollen in deionized water at strains less than 30%, mechanical recovery experiments were not performed on these samples.

Rheological Recoverability

The rheological recoverability of samples at 15% w/v nanoparticle concentration was also evaluated after colloidal gel fabrication (FIG. 33 and FIG. 34). The viscosity of the colloidal gels was recoverable after applying shear stress to the samples. These results suggested that surface chains could re-associate after disruption by shear force. After applying shear force, samples still had similar rheological behavior compared to the initial material which suggests recovery of the colloidal gel network after applying shear. Studies have shown a reduction in the viscosity of HA solutions after applying shear stress and HA has been found to degrade under shear. The slight drop in viscosity after the first sweep might be due to the degradation of HA or an insufficient delay between each run. The drop in viscosity was less after the second run (FIG. 34).

Physical Recoverability

Colloidal gels at 30% w/v nanoparticle concentration (17 kDa HA) formed a network after fabrication and after swelling in deionized water. The steps for performing this recoverability experiment are shown in FIG. 35. First, nanoparticles made from 17 kDa HA were used to make the colloidal gel at 30% w/v concentration. The colloidal gel was then swollen and equilibrated in deionized water for at least 24 hours. After swelling the sample, the colloidal gel was completely crushed. Then, the sample was transferred to a desiccator chamber and left to dry for at least 48 hours. Finally, the dried sample was again used to form a colloidal gel network at 30% w/v nanoparticle concentration in deionized water. A colloidal gel was again formed from the dried sample. These data suggested that dangling chains available on nanoparticles could entangle with each other even after colloidal gel destruction.

Colloidal gel networks were formed by addition of HA nanoparticles in deionized water. The type of polymer, molecular weight of polymer, and nanoparticle concentration significantly influenced nanoparticle-nanoparticle interactions and colloidal gel formation. Stable 3-D colloidal gel networks formed at 15%, 30%, and 45% concentrations when using nanoparticles fabricated with 17 kDa HA. At these concentrations, physical entanglement between 17 kDa HA nanoparticles with ‘hairy’ structure might be the reason for colloidal gel formation. After mixing nanoparticles with deionized water, dangling chains on the nanoparticles might entangle and interlock resulting in formation of stable colloidal gels. Colloidal gels did not form at 5% w/v when 17 kDa HA nanoparticles were used. This was likely due to insufficient nanoparticle concentration to achieve physical entanglement. On the other hand, HA nanoparticles made from 1500 kDa HA could not form colloidal gel networks even at high concentration probably due to the limited availability of dangling chains on these nanoparticles. Also, CS nanoparticles could not form colloidal gel networks probably due to the nature of CS and the absence of physical entanglement or ‘self-association’ in this highly charged GAG.

Swelling experiments suggested that colloidal gel networks were stable upon dilution in an excess of deionized water and in 0.1 M PBS. The results also indicated that the swelling ratio was dependent on nanoparticle concentration and the nature of the swelling solution. Increasing nanoparticle concentration may increase physical entanglement between nanoparticles reducing the swelling ratio. Moreover, the presence of salt in swelling solution is known to reduce hydrodynamic radius of HA and physical entanglement. As a result, the swelling ratio of the samples in 0.1 M PBS was lower than the swelling ratio of the samples in deionized water. A comparison of HA colloidal gels and classic HA hydrogels including HA crosslinked via divinyl sulfone (DVS), photocrosslinked methacrylated HA, dual-crosslinked HA (photo crosslinked and chemically crosslinked), and HA via crosslinked disulfide bond formation, showed that the swelling ratio of the colloidal gels was in the range of a maximum swelling ratio of classic gels (i.e. a low degree of crosslinking).

In addition, compression testing also suggested that the mechanical properties of colloidal gels (Young's and shear moduli) were influenced by nanoparticle concentration and sample condition. Increasing nanoparticle concentration made colloidal gels stiffer. Moreover, samples swollen in deionized water had greater Young's modulus compared to the samples after fabrication or to samples swollen in 0.1 M PBS. These behaviors may also hinge on the physical entanglement between nanoparticles which was influenced by nanoparticle concentration and sample condition. In addition, compression testing indicated that colloidal gels behaved as a softer material compared to classic HA hydrogels. Maximum Young's and shear moduli of the colloidal gels reached approximately 20 kPa; however, normal Young's and shear moduli values for HA crosslinked with DVS were in the range of several thousand kPa. Comparing the shear modulus of colloidal gels with photocrosslinked methacrylated HA (20<G<60 kPa) also showed that the colloidal gels had softer mechanical properties.

In other studies, nanoparticle concentration had an important effect on rheological behavior of colloidal gels. Increasing nanoparticle concentration increased storage modulus, loss modulus, complex modulus, and tan delta of the samples after fabrication. Moreover, viscosity of the samples upon mixing nanoparticles with deionized water increased by increasing nanoparticle concentration. The shear stress and viscosity of the colloidal gels increased up to a yield point by increasing the shear rate. After the yield point, viscosity of the samples decreased by increasing the shear rate indicative of shear thinning behavior for the colloidal gels. Viscoelasticity measurements also indicated that colloidal gels had a similar storage modulus, loss modulus, and complex modulus compared to a classic hydrogel. Previous viscoelasticity evaluation of HA crosslinked via DVS showed that the storage and loss moduli were in the range of several hundred to several thousand Pa and the colloidal gels had similar viscoelasticity.

Finally, recoverability experiments revealed the dynamic properties of colloidal gels. Mechanical experiments suggested that the height of the samples was recoverable to the initial height after compressing/decompressing samples independent of nanoparticle concentration. Rheological experiments also showed that the viscosity of the colloidal gels was recoverable after applying shear stress to the samples. These results support the notion that the interrupted physical entanglement between surface chains on nanoparticles could re-associate after removing shear force. A physical recoverability experiment showed that colloidal gels can be destroyed, dried, and reconstituted are still form a colloidal gel network.

In conclusion, colloidal gel networks were formed by mixing HA nanoparticles in deionized water. The type of polymer, molecular weight of polymer, and nanoparticle concentration significantly influenced nanoparticle-nanoparticle interactions and colloidal gel formation. Stable 3-D colloidal gel networks formed at 15%, 30%, and 45% concentrations when using nanoparticles fabricated with 17 kDa HA due to physical entanglement between nanoparticles. CS nanoparticles and nanoparticles made from 1500 kDa HA could not form colloidal gel networks probably due to limited availability of surface chains to mediate nanoparticle-nanoparticle interactions. Mechanical and rheological experiments showed that Young's modulus, shear modulus, viscosity, and viscoelasticity of colloidal gels were influenced by nanoparticle concentration and media type. Finally, recoverability experiments confirmed that colloidal gels had dynamic properties. Formation of stable 3-D colloidal gels using HA nanoparticles without any potentially toxic chemical reactions suggests a new approach for scaffold fabrication and tissue regeneration.

### Example 3

### Application of Hyaluronic Acid Nanoparticles in Colloidal Suspensions as a Potential Osteoarthritis Treatment

According to the present disclosure, HA nanoparticles were explored as a means to reduce the viscosity of viscosupplements using high molecular weight HA. Orthovisc® is made from uncrosslinked 1500 kDa HA and has the highest concentration of HA among the uncrosslinked viscosupplements (Table 1-5). In this study, HA nanoparticles were used to reduce the viscosity of HA solutions with similar molecular weight and concentration to Orthovisc®. Two experiments were designed to investigate the effect of nanoparticles on the rheological behavior (viscosity and viscoelasticity) of simulated Orthovisc®. A solution of 1500 kDa at 1.4% w/v was prepared as a model viscosupplement for osteoarthritis treatment. The effect of including nanoparticles at different ratios and changing the molecular weight of HA used for nanoparticle fabrication was evaluated rheologically.

Materials

Nanoparticles fabricated as previously described at 1× polymer concentration, 1:1 molar reactive site ratio, and three hours reaction time made from 17 kDa and 1500 kDa HA were used for the experiments.

Methods

Mixing HA Nanoparticles with HA Polymer to Reach Hyaluronic Acid Concentration Simulated Orthovisc® Formulation

To evaluate the effect of HA nanoparticles in a simulated Orthovisc® formulation, an overall HA concentration was set at 1.4% w/v which is the same concentration of hyaluronic acid (1500 kDa) in Orthovisc®. Two sets of samples were prepared by mixing nanoparticles made from 17 kDa HA or nanoparticles made from 1500 kDa HA with 1500 kDa HA polymer at different nanoparticle: polymer ratios to reach the final HA concentration of 1.4% w/v in deionized water. Polymer to nanoparticle ratios were selected as: 100:0, 75:25, 50:50, 25:75, and 0:100. After removing the dry polymer and nanoparticles from the freezer and equilibrating them at room temperature, samples were prepared using these ratios by mixing them in deionized water. Control samples were also prepared by mixing 1500 kDa HA polymer in deionized water at different concentrations (1.05%, 0.7%, and 0.35% w/v).

Using HA Nanoparticle Formulations at Different Concentrations

To evaluate the effect of nanoparticle concentration on the viscosity and viscoelasticity of HA nanoparticle suspensions, nanoparticles made of 1500 kDa HA were mixed at 1.4%, 5%, 10%, and 15% w/v concentrations with deionized water. 1500 kDa HA polymer at 1.4% w/v was also selected as a control (simulated Orthovisc®).

Viscosity Measurement

To investigate the viscosity of samples made as described above, an AR-G2 rheometer (TA Instruments, Delaware) equipped with a 20, 20 mm diameter cone-plate at 25° C. was employed. Similar to the procedure described above, after loading samples under the rheometer, shear stress and viscosity of the samples were measured over a shear rate sweep of 0.01-1000 s⁻¹.

Viscoelasticity Measurement

To evaluate the viscoelasticity of samples prepared as described above, dynamic oscillatory rheological measurements were performed using an AR-G2 rheometer (TA Instruments, Delaware) equipped with a 20, 20 mm diameter cone-plate at 25° C. Similar to the procedure described above, first, strain sweep experiments were performed to determine the limit of viscoelasticity, where the rheological properties are strain dependent. Then, the viscoelastic properties of colloidal suspensions were evaluated using a frequency sweep from 0.1 to 10 Hz at 1% strain, which was in the linear viscoelastic region. This range includes the physiological frequencies of the knee, ranging from 0.5 Hz (walk) to 3 Hz (running). All experiments were performed in triplicate.

Results

All formulations were prepared as described and tested to evaluate their viscosity and viscoelasticity (FIG. 36). A simulated Orthovisc® using a HA polymer solution (1500 kDa) at 1.4% w/v concentration was compared to a HA nanoparticle suspension (1500 kDa) at similar concentration. It was observed that the polymer solution was highly viscous; however, the nanoparticle suspension was more water-like. The nanoparticle formation obviously reduced the viscosity and increased the fluidity of HA at 1.4% w/v.

Viscosity Measurement: Mixing HA Nanoparticles with HA Polymer to Reach Hyaluronic Acid Concentration in the Orthovisc® Formulation

The rheological behavior of HA polymer/nanoparticle mixtures using 1500 kDa polymer and nanoparticles made from 17 kDa or 1500 kDa HA was explored. Increasing nanoparticle concentration reduced the shear stress and viscosity of the polymer/nanoparticle mixtures independent of the HA molecular weight used for nanoparticle fabrication. The highest shear rate and viscosity values were observed for the sample with 100% HA polymer. On the other hand, the lowest shear rate and viscosity values were for the samples with 100% HA nanoparticles made from either 17 kDa or 1500 kDa HA.

Comparing polymer/nanoparticle mixtures made from either 17 kDa or 1500 kDa HA nanoparticles showed that samples containing 17 kDa HA nanoparticles had higher viscosity than samples made from 1500 kDa HA nanoparticles at all polymer: nanoparticle ratios (FIG. 41). These results suggested that nanoparticles made from 17 kDa HA might interact more with 1500 kDa polymer chains resulting in greater viscosity. This interaction might be the result of dangling chains on the surface of 17 kDa nanoparticles. This hairy structure might facilitate entanglement with polymer chains resulting in greater viscosity values compared to the samples made from 1500 kDa HA nanoparticles at similar polymer: nanoparticle ratios. Lower viscosity samples made from 1500 kDa HA nanoparticles might be due to inhibiting polymer chain entanglement between HA molecules in solution. These data could also support the formation of colloidal gels using 17 kDa HA nanoparticles and the formation of paste-like materials using 1500 kDa HA nanoparticles.

In addition, the difference in initial viscosity of samples using 17 kDa or 1500 kDa HA nanoparticles increased by increasing the concentration of HA polymer in the samples. The samples at a 75:25 ratio had the greatest initial viscosity difference comparing the two nanoparticle types (FIG. 41). Increasing nanoparticle concentration decreased this viscosity difference as well as the overall viscosity of the samples. This might be due to more polymer-nanoparticle interaction and physical entanglement in the samples with higher polymer concentrations. Therefore, the viscosity of polymer/nanoparticle mixtures could be controlled using nanoparticles made from HA with different molecular weights.

Formulations containing 1500 kDa HA nanoparticles had viscosity values closer to the control samples at different polymer:nanoparticle ratios. Increasing the concentration of nanoparticles showed that the difference between the initial viscosity of the formulations and controls became lower. These results could suggest that the entanglement of free surface chains on nanoparticles was more dependent on the concentration of free polymer in the suspension. Moreover, the absence of sufficient dangling chains on 1500 kDa HA nanoparticles led to low interaction with either polymer chains or nanoparticles, thus reducing the viscosity the mixtures.

Using HA Nanoparticle Formulations at Different Concentrations

In the previous experiment, the viscosity of suspensions made from 17 kDa and 1500 kDa HA nanoparticles was lower than the viscosity of HA polymer solution (1500 kDa) at 1.4% w/v. Here, the effect of increasing nanoparticle (1500 kDa HA) concentration from 1.4% w/v up to 15% w/v on the shear stress and viscosity of the nanoparticle suspensions was investigated. The formation of colloidal systems from 1500 kDa HA nanoparticles (1.4% w/v up to 45% w/v) was observed (FIG. 8). Samples at 30% w/v and 45% w/v nanoparticle concentrations formed paste-like materials and the rheological properties could not be measured due to high viscosity of these samples. Therefore, nanoparticle concentrations up to 15% w/v were used for this experiment (FIGS. 42 and 43). The rheological behavior of HA nanoparticle (1500 kDa) samples at 1.4%, 5%, 10%, and 15% w/v concentrations were compared to HA polymer solution (1500 kDa) at 1.4% w/v.

Increasing nanoparticle concentration increased shear stress and viscosity as shear rate increased. These results indicated that even at 15% w/v nanoparticle concentration, shear stress and viscosity of the sample were still lower than the shear stress and viscosity of 1.4% w/v HA in solution. Nanoparticle formation not only reduced the viscosity of HA, but also it may also be used to increase the concentration of HA in formulations. Therefore, higher HA concentrations can be injected into the body with only a small increase in the formulation viscosity.

Viscoelasticity Measurement: Mixing HA Nanoparticles with HA Polymer to Reach Hyaluronic Acid Concentration in Orthovisc® Formulation

The viscoelastic behavior of polymer/nanoparticle mixtures using 1500 kDa polymer and nanoparticles made from 17 kDa and 1500 kDa HA was evaluated (FIGS. 44 to 49). Increasing nanoparticle concentration lowered storage, loss, and complex moduli of the polymer/nanoparticle mixtures independent of the HA molecular weight used for the nanoparticles. The highest viscoelastic values were observed for the samples with 100% HA polymer. In contrast, the lowest viscoelasticity was found for the samples with 100% HA nanoparticles made from either 17 kDa or 1500 kDa HA.

Polymer/nanoparticle mixtures made using 17 kDa HA nanoparticles showed a higher storage, loss, and complex moduli when compared to the samples made from 1500 kDa HA nanoparticles at all polymer: nanoparticle ratios (FIGS. 50, 51, and 52). Similar to viscosity measurements, these results also suggested that nanoparticles made from 17 kDa HA might interact more with HA polymer chains in solution resulting in greater viscoelasticity for the samples made from 17 kDa HA nanoparticles. The hairy structure of nanoparticles made from 17 kDa HA might physically entangle with HA polymer chains resulting in greater viscoelasticity values compared to 1500 kDa HA nanoparticles. On the other hand, nanoparticles made from 1500 kDa HA may behave more like a hard sphere or may even inhibit HA polymer-polymer interaction in solution. Formulations containing 1500 kDa HA nanoparticles had the viscoelasticity values closer to the control samples at different polymer:nanoparticle ratios. Increasing the concentration of nanoparticles decreased the difference between the viscoelasticity of the formulations and controls.

Using HA Nanoparticle Formulations at Different Concentrations

The effect of increasing nanoparticle (1500 kDa HA) concentration from 1.4% w/v up to 15% w/v on the viscoelasticity of the nanoparticle suspensions was also evaluated (FIGS. 53, 54, and 55). Increasing nanoparticle concentration increased storage, loss, and complex moduli of the colloidal suspensions. Therefore, viscoelastic properties of colloidal suspensions could be controlled by changing the nanoparticle concentration in these formulations when compared to 1.4% polymer solution.

Our study showed that increasing nanoparticle concentration in polymer/nanoparticle mixtures (HA concentration of 1.4% w/v) reduced viscosity and viscoelasticity of the samples. This decrease was influenced by nanoparticles which were synthesized from HA with different molecular weights. The surface structure of nanoparticles, which was dependent on molecular weight of HA used for nanoparticle fabrication, might change the rheological properties of colloidal suspensions. Nanoparticles made from 17 kDa HA, which were presumed to have ‘hairy’ surface structure, seemed to have more polymer-nanoparticle interaction and physical entanglement in polymer/nanoparticle mixtures compared to nanoparticles made from 1500 kDa HA. These interactions might be the reason for greater viscosity and viscoelasticity in the mixtures made from 17 kDa HA nanoparticles compared to the mixtures made from 1500 kDa HA nanoparticles at similar polymer/nanoparticle ratios. Rheological studies also showed that even 1500 kDa HA nanoparticles interacted with HA polymer influencing viscosity and viscoelasticity. This interaction depended on the polymer/nanoparticle ratio. At higher polymer concentrations, more physical interactions between nanoparticles (17 kDa or 1500 kDa) must have yielded a greater effect on rheological properties of samples. By increasing nanoparticle concentration in the formulations with 1500 kDa nanoparticles, the viscosity and viscoelasticity approached the viscosity and the viscoelasticity of control samples containing only polymer. Therefore, the rheological properties of the viscosupplements can be controlled via the type and the concentration of nanoparticles mixed into the formulation.

Application of nanoparticles to enhance rheological properties of fluids and to form formation of nanocomposites has been reported. One study suggested that the presence of a small amount of nanoparticles with large aspect ratio improved the fluidity of polymeric solutions under low shear rate. On the other hand, addition of a large amount of nanoparticles reduced the aspect ratio by aggregation and constrained polymer segment motion in solutions. Other studies also reported the application of inorganic nanoparticles such as clay nanoparticles in ophthalmic and otic pharmaceutical formulations to modify the rheological properties of the compositions to enhance the viscosity, flow characteristics, or lubricity of the products. The studies showed that nanoparticles could enhance shear thinning behavior and fluidity. These reports and others confirmed that nanoparticles can modify the rheological properties of solutions but the effect of surface structure of nanoparticles to control rheological properties of solutions has not been clearly reported. Here, the rheological properties of viscosupplements could be controlled via nanoparticles. Different types of nanoparticles (17 kDa or 1500 kDa HA) at different concentrations can be added to the viscosupplements to control the viscosity and viscoelasticity.

Moreover, increasing HA nanoparticle (1500 kDa) concentration increased both viscosity and viscoelasticity of HA polymer solutions. Even at relatively high (15% w/v) nanoparticle concentration, the viscosity of the samples was lower than the control sample simulating Orthovisc® (1.4% w/v HA solution). At higher nanoparticle concentrations (30% and 45% w/v), formation of paste-like material was observed. Rheological measurement at these nanoparticle concentrations was difficult due to the high viscosity of these samples. Finally, nanoparticle formation not only reduced the viscosity of HA suspensions but also could be used to increase the total concentration of HA in the formulation. As a result, colloidal suspensions can be used to increase the HA concentration with a small increase in the viscosity of HA viscosupplements.

Particle fabrication is one of the techniques used to reduce the viscosity of a solution. Currently, crystallization, phase separation, and complexation have also been reported to enhance the viscosity and increase injectability of protein solutions, specifically monoclonal antibodies at high doses. Such particle fabrication approaches packed the proteins, reduced the concentration of free protein in solution, and prevented interactions which typically caused an increase in viscosity. With nanoparticle fabrication, the viscosity of HA viscosupplements can be reduced and the injectability enhanced in addition to increasing the concentration of HA in formulation.

In conclusion, the ability of hyaluronic acid nanoparticles to modify the viscosity and viscoelasticity of a simulated viscosupplement was evaluated. Increasing nanoparticle concentration in polymer/nanoparticle mixtures (overall HA concentration of 1.4% w/v) reduced viscosity and viscoelasticity of the samples. The type of nanoparticles (17 kDa or 1500 kDa) influenced the nanoparticle-polymer interactions and controlled the rheological properties of the suspensions. In addition, the viscosity of suspensions that included 1500 kDa HA nanoparticles was lower when compared to simulated viscosupplement. The viscosity of suspensions containing 15% w/v of nanoparticles was also lower than the viscosity of simulated viscosupplement (1.4 w/v HA). Therefore, nanoparticle formations not only reduced the viscosity of HA suspensions but facilitated an increase in the HA concentration with minimal increase in the viscosity of viscosupplements in an effort to enhance injectability.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

### REFERENCES

1. Kurisawa M, Chung J, Yang Y, Gao S, Uyama H. Injectable biodegradable hydrogels composed of hyaluronic acid-tyramine conjugates for drug delivery and tissue engineering. Chemical communications. 2005; 2005(34):4312-4.
2. Garg H G, Hales C A. Chemistry and biology of hyaluronan: Elsevier Science; 2004.
3. Necas J, Bartosikova L, Brauner P, Kolar J. Hyaluronic acid (hyaluronan): a review. Veterinarni Medicina. 2008; 53(8):397-411.
4. Falcone S, Palmeri D, Berg R, editors. Biomedical applications of hyaluronic acid2006: ACS Publications.
5. Zheng Shu X, Liu Y, Palumbo F, Luo Y, Prestwich G. In situ crosslinkable hyaluronan hydrogels for tissue engineering. Biomaterials. 2004; 25(7-8):1339-48.
6. Vejlens L. Glycosaminoglycans of human bone tissue. Calcified Tissue International. 1971; 7(1):175-90.
7. Dumitriu S. Polymeric biomaterials. New York: Marcel Dekker Inc.; 2002.
8. Segura T, Anderson B, Chung P, Webber R, Shull K, Shea L. Crosslinked hyaluronic acid hydrogels: a strategy to functionalize and pattern. Biomaterials. 2005; 26(4):359-71.
9. Toole B. Hyaluronan: from extracellular glue to pericellular cue. Nature Reviews Cancer. 2004; 4(7):528-39.
10. Cai S, Xie Y, Bagby T, Cohen M, Forrest M. Intralymphatic chemotherapy using a hyaluronan-cisplatin conjugate. The Journal of surgical research. 2008; 147(2):247.
11. UNDERHILL C. CD44: the hyaluronan receptor. Journal of Cell Science. 1992; 103(2):293.
12. Eng D, Caplan M, Preul M, Panitch A. Hyaluronan Scaffolds—A Balance between Backbone Functionalization and Bioactivity. Acta Biomaterialia. 2010.
13. Kim J, Kim I, Cho T, Lee K, Hwang S, Tae G, et al. Bone regeneration using hyaluronic acid-based hydrogel with bone morphogenic protein-2 and human mesenchymal stem cells. Biomaterials. 2007; 28(10):1830-7.
14. Luo Y, Kirker K, Prestwich G. Cross-linked hyaluronic acid hydrogel films: new biomaterials for drug delivery. Journal of controlled release. 2000; 69(1):169-84.
15. Lee K, Mooney D. Hydrogels for tissue engineering. Chem Rev. 2001; 101(7):1869-80.
16. Drury J, Mooney D. Hydrogels for tissue engineering: scaffold design variables and applications. Biomaterials. 2003; 24(24):4337-51.
17. Volpi N, Schiller J, Stem R, Soltes L. Role, metabolism, chemical modifications and applications of hyaluronan. Current medicinal chemistry. 2009; 16(14): 1718-45.
18. Burdick J A, Prestwich G D. Hyaluronic Acid Hydrogels for Biomedical Applications. Advanced Materials.
19. Whelan J. The Biology of hyaluronan: John Wiley & Sons; 1989.
20. Sheehan J, Arundel C, Phelps C. Effect of the cations sodium, potassium and calcium on the interaction of hyaluronate chains: a light scattering and viscometric study. International Journal of Biological Macromolecules. 1983; 5(4):222-8.
21. Toole B P, editor. Hyaluronan in morphogenesis 2001: Elsevier.
22. Toole B P, Wight T N, Tammi M I. Hyaluronan-cell interactions in cancer and vascular disease. Journal of Biological Chemistry. 2002; 277(7):4593.
23. Noble P W. Hyaluronan and its catabolic products in tissue injury and repair. Matrix biology. 2002; 21(1):25-9.
24. Toole B. Hyaluronan in morphogenesis. Journal of internal medicine. 1997; 242(1):35-40.
25. McKee C M, Penno M B, Cowman M, Burdick M D, Strieter R M, Bao C, et al. Hyaluronan (HA) fragments induce chemokine gene expression in alveolar macrophages. The role of H A size and CD44. Journal of Clinical Investigation. 1996; 98(10):2403.
26. Oertli B, Fan X, Wiithrich R P. Characterization of CD44-mediated hyaluronan binding by renal tubular epithelial cells. Nephrology Dialysis Transplantation. 1998; 13(2):271.
27. Chen W Y J, Abatangelo G. Functions of hyaluronan in wound repair. Wound Repair and Regeneration. 1999; 7(2):79-89.
28. Lee J Y, Spicer A P. Hyaluronan: a multifunctional, megaDalton, stealth molecule. Current Opinion in Cell Biology. 2000; 12(5):581-6.
29. Cowman M K, Matsuoka S. Experimental approaches to hyaluronan structure. Carbohydrate research. 2005; 340(5):791-809.
30. Stern R, Kogan G, Jedrzejas M J, Soltés L. The many ways to cleave hyaluronan. Biotechnology advances. 2007; 25(6):537-57.
31. Soltés L, Mendichi R, Kogan G, Schiller J, Stankovska M, Amhold J. Degradative action of reactive oxygen species on hyaluronan. Biomacromolecules. 2006; 7(3):659-68.
32. Arrich J, Piribauer F, Mad P, Schmid D, Klaushofer K, Müllner M. Intra-articular hyaluronic acid for the treatment of osteoarthritis of the knee: systematic review and meta-analysis. Canadian Medical Association Journal. 2005; 172(8):1039.
33. Goldberg V, Buckwalter J. Hyaluronans in the treatment of osteoarthritis of the knee: evidence for disease-modifying activity. Osteoarthritis and cartilage. 2005; 13(3):216-24.
34. Migliore A, Granata M. Intra-articular use of hyaluronic acid in the treatment of osteoarthritis. Clinical interventions in aging. 2008; 3(2):365.
35. Allison D D, Grande-Allen K J. Review. Hyaluronan: a powerful tissue engineering tool. Tissue Engineering. 2006; 12(8):2131-40.
36. Jiang D, Liang J, Noble P W. Hyaluronan in tissue injury and repair. Annu Rev Cell Dev Biol. 2007; 23:435-61.
37. Tomihata K, Ikada Y. Crosslinking of hyaluronic acid with water soluble carbodiimide. Journal of biomedical materials research. 1997; 37(2):243-51.
38. Vercruysse K P, Marecak D M, Marecek J F, Prestwich G D. Synthesis and in vitro degradation of new polyvalent hydrazide cross-linked hydrogels of hyaluronic acid. Bioconjugate chemistry. 1997; 8(5):686-94.
39. Luo Y, Kirker K R, Prestwich G D. Cross-linked hyaluronic acid hydrogel films: new biomaterials for drug delivery. Journal of controlled release. 2000; 69(1):169-84.
40. Liu Y, Zheng Shu X, Prestwich G D. Biocompatibility and stability of disulfide-crosslinked hyaluronan films. Biomaterials. 2005; 26(23):4737-46.
41. Baier Leach J, Bivens K A, Patrick Jr C W, Schmidt C E. Photocrosslinked hyaluronic acid hydrogels: natural, biodegradable tissue engineering scaffolds. Biotechnology and bioengineering. 2003; 82(5):578-89.
42. Burdick J A, Chung C, Jia X, Randolph M A, Langer R. Controlled degradation and mechanical behavior of photopolymerized hyaluronic acid networks. Biomacromolecules. 2005; 6(1):386-91.
43. Cen L, Neoh K, Li Y, Kang E. Assessment of in vitro bioactivity of hyaluronic acid and sulfated hyaluronic acid functionalized electroactive polymer. Biomacromolecules. 2004; 5(6):2238-46.
44. Collier J H, Camp J P, Hudson T W, Schmidt C E. Synthesis and characterization of polypyrrole-hyaluronic acid composite biomaterials for tissue engineering applications. Journal of biomedical materials research. 2000; 50(4):574-84.
45. Aigner J, Tegeler J, Hutzler P, Campoccia D, Pavesio A, Hammer C, et al. Cartilage tissue engineering with novel nonwoven structured biomaterial based on hyaluronic acid benzyl ester. Journal of biomedical materials research. 1998; 42(2):172-81.
46. Ramamurthi A, Vesely I. Evaluation of the matrix-synthesis potential of crosslinked hyaluronan gels for tissue engineering of aortic heart valves. Biomaterials. 2005; 26(9):999-1010.
47. Ibrahim S, Kang Q K, Ramamurthi A. The impact of hyaluronic acid oligomer content on physical, mechanical, and biologic properties of divinyl sulfone crosslinked hyaluronic acid hydrogels. Journal of Biomedical Materials Research Part A. 2010; 94(2):355-70.
48. Zheng Shu X, Liu Y, Palumbo F S, Luo Y, Prestwich G D. In situ crosslinkable hyaluronan hydrogels for tissue engineering. Biomaterials. 2004; 25(7-8):1339-48.
49. Yeo Y, Highley C B, Bellas E, Ito T, Marini R, Langer R, et al. In situ cross-linkable hyaluronic acid hydrogels prevent post-operative abdominal adhesions in a rabbit model. Biomaterials. 2006; 27(27):4698-705.
50. Yeo Y, Ito T, Bellas E, Highley C B, Marini R, Kohane D S. In situ cross-linkable hyaluronan hydrogels containing polymeric nanoparticles for preventing postsurgical adhesions.
Annals of surgery. 2007; 245(5):819.
51. Tezel A, Fredrickson G H. The science of hyaluronic acid dermal fillers. Journal of Cosmetic and Laser Therapy. 2008; 10(1):35-42.
52. Sadick N S, Karcher C, Palmisano L. Cosmetic dermatology of the aging face. Clinics in Dermatology. 2009; 27(3):S3-S12.
53. Fisher G J, Varani J, Voorhees J J. Looking older: fibroblast collapse and therapeutic implications. Archives of dermatology. 2008; 144(5):666.
54. Sánchez-Carpintero I, Candelas D, Ruiz-Rodriguez R. Dermal Fillers: Types, Indications, and Complications. Actas Dermo-Sifiliogrificas (English Edition). 2010; 101(5):381-93.
55. CHRISTENSEN L H. Host tissue interaction, fate, and risks of degradable and nondegradable gel fillers. Dermatologic Surgery. 2009; 35:1612-9.
56. Andre P. New trends in face rejuvenation by hyaluronic acid injections. Journal of Cosmetic Dermatology. 2008; 7(4):251-8.
57. Brandt F S, Cazzaniga A. Hyaluronic acid gel fillers in the management of facial aging.
Clinical interventions in aging. 2008; 3(1):153.
58. Gold M H. Use of hyaluronic acid fillers for the treatment of the aging face. Clinical interventions in aging. 2007; 2(3):369.
59. Buck I, Donald W, Alam M, Kim J. Injectable fillers for facial rejuvenation: a review. Journal of Plastic, Reconstructive & Aesthetic Surgery. 2009; 62(1): 11-8.
60. Monheit G D, Prather C L. Hyaluronic acid fillers for the male patient. Dermatologic therapy. 2007; 20(6):394-406.
61. KABLIK J, MONHEIT G D, Y U L, CHANG G, GERSHKOVICH J. Comparative physical properties of hyaluronic acid dermal fillers. Dermatologic Surgery. 2009; 35:302-12.
62. Moreland L W. Intra-articular hyaluronan (hyaluronic acid) and hylans for the treatment of osteoarthritis: mechanisms of action. Arthritis Research and Therapy. 2003; 5(2):54-67.
63. Yaszemski M J. Tissue engineering and novel delivery systems: CRC Press; 2003.
64. Conrad B P. The effects of glucosamine and chondroitin on the viscosity of synovial fluid in patients with osteoarthritis: Citeseer; 2001.
65. Altman R, Moskowitz R. Intraarticular sodium hyaluronate (Hyalgan) in the treatment of patients with osteoarthritis of the knee: a randomized clinical trial. Hyalgan Study Group. The Journal of rheumatology. 1998; 25(11):2203.
66. Maheu E, Ayral X, Dougados M. A hyaluronan preparation (500-730 kDa) in the treatment of osteoarthritis: a review of clinical trials with Hyalgan. International journal of clinical practice. 2002; 56(10):804.
67. Watterson J R, Esdaile J M. Viscosupplementation: therapeutic mechanisms and clinical potential in osteoarthritis of the knee. Journal of the American Academy of Orthopaedic Surgeons. 2000; 8(5):277.
68. CARE FONP. Hyaluronic acid products for osteoarthritis of the knee. CPJ. 2007; 140(3):195.
69. Fam H, Bryant J, Kontopoulou M. Rheological properties of synovial fluids. Biorheology. 2007; 44(2):59-74.
70. Balazs E A, Denlinger J L. Viscosupplementation: A new concept in the treatment of osteoarthritis. The Journal of rheumatology Supplement. 1993; 39:3.
71. Sun S F, Chou Y J, Hsu C W, Chen W L. Hyaluronic acid as a treatment for ankle osteoarthritis. Current reviews in musculoskeletal medicine. 2009; 2(2):78-82.
72. Leopold S S, Warme W J, Pettis P D, Shott S. Increased frequency of acute local reaction to intra-articular hylan GF-20 (synvisc) in patients receiving more than one course of treatment. The Journal of bone and joint surgery American volume. 2002; 84(9):1619.
73. Kotevoglu N, Iy bozkurt P C, H z O, Toktas H, Kuran B. A prospective randomised controlled clinical trial comparing the efficacy of different molecular weight hyaluronan solutions in the treatment of knee osteoarthritis. Rheumatology international. 2006; 26(4):325-30.
74. Cegnar M, KERC J. Self-assembled Polyelectrolyte Nanocomplexes of Alginate, Chitosan and Ovalbumin. Acta chimica slovenica. 2010; 57(2):431-41.
75. Kamiya N, Klibanov A M. Controlling the rate of protein release from polyelectrolyte complexes. Biotechnology and bioengineering. 2003; 82(5):590-4.
76. Hu Z, Xia X, Tang L. Process for synthesizing oil and surfactant-free hyaluronic acid nanoparticles and microparticles. US Patent App. 20,060/040,892; 2004.
77. Vauthier C, Bouchemal K. Methods for the preparation and manufacture of polymeric nanoparticles. Pharmaceutical research. 2009; 26(5):1025-58.
78. Fakhari A, Baoum A, Siahaan T J, Le K B, Berkland C. Controlling ligand surface density optimizes nanoparticle binding to ICAM 1. Journal of pharmaceutical sciences. 2010.
79. Baoum A, Xie S X, Fakhari A, Berkland C. “Soft” Calcium Crosslinks Enable Highly Efficient Gene Transfection Using TAT Peptide. Pharmaceutical research. 2009; 26(12):2619-29.
80. Chittasupho C, Manikwar P, Krise J P, Siahaan T J, Berkland C. cIBR effectively targets nanoparticles to LFA-1 on acute lymphoblastic T cells. Molecular pharmaceutics. 2009; 7(1):146-55.
81. Chittasupho C, Xie S X, Baoum A, Yakovleva T, Siahaan T J, Berkland C J. ICAM-1 targeting of doxorubicin-loaded PLGA nanoparticles to lung epithelial cells. European journal of pharmaceutical sciences. 2009; 37(2):141-50.
82. Zhang N, Chittasupho C, Duangrat C, Siahaan T J, Berkland C. PLGA Nanoparticle-Peptide Conjugate Effectively Targets Intercellular Cell-Adhesion Molecule-1. Bioconjugate chemistry. 2007; 19(1):145-52.
83. Wang Q, Jamal S, Detamore M S, Berkland C. PLGA chitosan/PLGA alginate nanoparticle blends as biodegradable colloidal gels for seeding human umbilical cord mesenchymal stem cells. Journal of Biomedical Materials Research Part A. 2011.
84. Wang Q, Wang J, Lu Q, Detamore M S, Berkland C. Injectable PLGA based colloidal gels for zero-order dexamethasone release in cranial defects. Biomaterials. 2010; 31(18):4980-6.
85. Wang Q, Wang L, Detamore M S, Berkland C. Biodegradable colloidal gels as moldable tissue engineering scaffolds. Advanced Materials. 2008; 20(2):236-9.
86. Jeong Y I, Kim S T, Jin S G, Ryu H H, Jin Y H, Jung T Y, et al. Cisplatin incorporated hyaluronic acid nanoparticles based on ion complex formation. Journal of pharmaceutical sciences. 2008; 97(3):1268-76.
87. Han S Y, Han H S, Lee S C, Kang Y M, Kim I S, Park J H. Mineralized hyaluronic acid nanoparticles as a robust drug carrier. J Mater Chem. 2011; 21(22):7996-8001.
88. Choi K Y, Chung H, Min K H, Yoon H Y, Kim K, Park J H, et al. Self-assembled hyaluronic acid nanoparticles for active tumor targeting. Biomaterials. 2010; 31(1):106-14.
89. Liu Y, Sun J, Cao W, Yang J, Lian H, Li X, et al. Dual targeting folate-conjugated hyaluronic acid polymeric micelles for paclitaxel delivery. International journal of pharmaceutics. 2011.
90. Cho H J, Yoon H Y, Koo H, Ko S H, Shim J S, Lee J H, et al. Self-assembled nanoparticles based on hyaluronic acid-ceramide (HA-CE) and Pluronic® for tumor-targeted delivery of docetaxel. Biomaterials. 2011.
91. Yang X, Kootala S, Hilborn J, Ossipov D A. Preparation of hyaluronic acid nanoparticles via hydrophobic association assisted chemical cross-linking—an orthogonal modular approach. Soft Matter. 2011.
92. Kyyronen K, Hume L, Benedetti L, Urtti A, Topp E, Stella V. Methylprednisolone esters of hyaluronic acid in ophthalmic drug delivery: in vitro and in vivo release studies. International journal of pharmaceutics. 1992; 80(1-3):161-9.
93. Hu Z, Xia X, Tang L. Process for synthesizing oil and surfactant-free hyaluronic acid nanoparticles and microparticles. U.S. Pat. No. 7,601,704; Filed Nov. 3, 2004.
94. Bouhadir K H, Hausman D S, Mooney D J. Synthesis of cross-linked poly(aldehyde guluronate) hydrogels. Polymer. 1999; 40(12):3575-84.
95. Lee K Y, Bouhadir K H, Mooney D J. Degradation behavior of covalently cross-linked poly(aldehyde guluronate) hydrogels. Macromolecules. 2000; 33(1):97-101.
96. Yang T F, Chen C N, Chen M C, Lai C H, Liang H F, Sung H W. Shell-crosslinked Pluronic L121 micelles as a drug delivery vehicle. Biomaterials. 2007; 28(4):725-34.
97. Izdebski J, Kunce D. Evaluation of carbodiimides using a competition method. Journal of Peptide Science. 1997; 3(2):141-4.
98. Gauthier M A, Klok H A. Peptide/protein-polymer conjugates: synthetic strategies and design concepts. Chem Commun. 2008(23):2591-611.
99. Yang H, Lopina S T. Penicillin V-conjugated PEG-PAMAM star polymers. Journal of Biomaterials Science, Polymer Edition. 2003; 14(10):1043-56.
100. Wang Q, Wang J, Lu Q, Detamore M S, Berkland C. Injectable PLGA based colloidal gels for zero-order dexamethasone release in cranial defects. Biomaterials. 2010; 31(18):4980-6.
101. Wang Q, Wang L, Detamore M S, Berkland C. Biodegradable colloidal gels as moldable tissue engineering scaffolds. Advanced Materials. 2008; 20(2):236-9.
102. Kretlow J D, Klouda L, Mikos A G. Injectable matrices and scaffolds for drug delivery in tissue engineering. Advanced drug delivery reviews. 2007; 59(4-5):263-73.
103. Yeo Y, Ito T, Bellas E, Highley C B, Marini R, Kohane D S. In situ cross-linkable hyaluronan hydrogels containing polymeric nanoparticles for preventing postsurgical adhesions. Annals of surgery. 2007; 245(5):819.
104. Tan H, Marra K G. Injectable, biodegradable hydrogels for tissue engineering applications. Materials. 2010; 3(3):1746-67.
105. Gutowska A, Jeong B, Jasionowski M. Injectable gels for tissue engineering. The Anatomical Record. 2001; 263(4):342-9.
106. Wang Q, Jamal S, Detamore M S, Berkland C. PLGA chitosan/PLGA alginate nanoparticle blends as biodegradable colloidal gels for seeding human umbilical cord mesenchymal stem cells. Journal of Biomedical Materials Research Part A. 2011.
107. Berkland C, Wang Q. Biodegradable Colloidal Gels as Moldable Tissue Engineering Scaffolds. Google Patents; 2008.
108. DeKosky B J, Dormer N H, Ingavle G C, Roatch C H, Lomakin J, Detamore M S, et al. Hierarchically designed agarose and poly(ethylene glycol) interpenetrating network hydrogels for cartilage tissue engineering. Tissue Engineering Part C: Methods. 2010; 16(6):1533-42.
109. Zawko S A, Suri S, Truong Q, Schmidt C E. Photopatterned anisotropic swelling of dual-crosslinked hyaluronic acid hydrogels. Acta Biomaterialia. 2009; 5(1):14-22.
110. Segura T, Anderson B C, Chung P H, Webber R E, Shull K R, Shea L D. Crosslinked hyaluronic acid hydrogels: a strategy to functionalize and pattern. Biomaterials. 2005; 26(4):359-71.
111. Fransson J, Espander Jansson A. Local tolerance of subcutaneous injections. Journal of pharmacy and pharmacology. 1996; 48(10):1012-5.
112. Gatej I, Popa M, Rinaudo M. Role of the pH on hyaluronan behavior in aqueous solution. Biomacromolecules. 2005; 6(1):61-7.
113. Dornhofer W, Embrechts E. Injection solution for intramuscular and subcutaneous administration to animals. Google Patents; 1998.
114. Garg H G, Hales C A. Chemistry and biology of hyaluronan: Elsevier Science; 2004.
115. Barbucci R, Lamponi S, Borzacchiello A, Ambrosio L, Fini M, Torricelli P, et al. Hyaluronic acid hydrogel in the treatment of osteoarthritis. Biomaterials. 2002; 23(23):4503-13.
116. Ghosh K, Shu X Z, Mou R, Lombardi J, Prestwich G D, Rafailovich M H, et al. Rheological characterization of in situ cross-linkable hyaluronan hydrogels. Biomacromolecules. 2005; 6(5):2857-65.
117. Fam H, Kontopoulou M, Bryant J. Effect of concentration and molecular weight on the rheology of hyaluronic acid/bovine calf serum solutions. Biorheology. 2009; 46(1):31-43.
118. Zhao X. Synthesis and characterization of a novel hyaluronic acid hydrogel. Journal of Biomaterials Science, Polymer Edition. 2006; 17(4):419-33.
119. \check{S}oltés L, Mendichi R, Kogan G, Schiller J, Stankovska M, Arnhold J. Degradative action of reactive oxygen species on hyaluronan. Biomacromolecules. 2006; 7(3):659-68.
120. Stern R, Kogan G, Jedrzejas M J, Soltés L. The many ways to cleave hyaluronan. Biotechnology advances. 2007; 25(6):537-57.
121. Volpi N, Schiller J, Stem R, Soltes L. Role, metabolism, chemical modifications and applications of hyaluronan. Current medicinal chemistry. 2009; 16(14): 1718-45.
122. Ibrahim S, Kang Q K, Ramamurthi A. The impact of hyaluronic acid oligomer content on physical, mechanical, and biologic properties of divinyl sulfone crosslinked hyaluronic acid hydrogels. Journal of Biomedical Materials Research Part A. 2010; 94(2):355-70.
123. Bencherif S A, Srinivasan A, Horkay F, Hollinger J O, Matyjaszewski K, Washburn N R. Influence of the degree of methacrylation on hyaluronic acid hydrogels properties. Biomaterials. 2008; 29(12):1739-49.
124. Jha A K, Hule R A, Jiao T, Teller S S, Clifton R J, Duncan R L, et al. Structural analysis and mechanical characterization of hyaluronic acid-based doubly cross-linked networks. Macromolecules. 2009; 42(2):537-46.
125. Shu X Z, Liu Y, Luo Y, Roberts M C, Prestwich G D. Disulfide cross-linked hyaluronan hydrogels. Biomacromolecules. 2002; 3(6):1304-11.
126. Barbucci R, Lamponi S, Borzacchiello A, Ambrosio L, Fini M, Torricelli P, et al. Hyaluronic acid hydrogel in the treatment of osteoarthritis. Biomaterials. 2002; 23(23):4503-13.
127. Conrad B P. The effects of glucosamine and chondroitin on the viscosity of synovial fluid in patients with osteoarthritis: Citeseer; 2001
128. Singh G, Miller J D, Lee F H, Pettitt D, Russell M W. Prevalence of cardiovascular disease risk factors among US adults with self-reported osteoarthritis: data from the Third National Health and Nutrition Examination Survey. population.7:17.
129. Yaszemski M J. Tissue engineering and novel delivery systems: CRC Press; 2003.
130. Watterson J R, Esdaile J M. Viscosupplementation: therapeutic mechanisms and clinical potential in osteoarthritis of the knee. Journal of the American Academy of Orthopaedic Surgeons. 2000; 8(5):277.
131. Altman R, Moskowitz R. Intraarticular sodium hyaluronate (Hyalgan) in the treatment of patients with osteoarthritis of the knee: a randomized clinical trial. Hyalgan Study Group. The Journal of rheumatology. 1998; 25(11):2203.
132. Maheu E, Ayral X, Dougados M. A hyaluronan preparation (500-730 kDa) in the treatment of osteoarthritis: a review of clinical trials with Hyalgan. International journal of clinical practice. 2002; 56(10):804.
133. Ghosh K, Shu X Z, Mou R, Lombardi J, Prestwich G D, Rafailovich M H, et al. Rheological characterization of in situ cross-linkable hyaluronan hydrogels. Biomacromolecules. 2005; 6(5):2857-65.
134. Fam H, Kontopoulou M, Bryant J. Effect of concentration and molecular weight on the rheology of hyaluronic acid/bovine calf serum solutions. Biorheology. 2009; 46(1):31-43.
135. Zhao X. Synthesis and characterization of a novel hyaluronic acid hydrogel. Journal of Biomaterials Science, Polymer Edition. 2006; 17(4):419-33.
136. Yin H, Mo D, Chen D. Orientation behavior of attapulgite nanoparticles in poly(acrylonitrile)/attapulgite solutions by rheological analysis. Journal of Polymer Science Part B: Polymer Physics. 2009; 47(10):945-54.
137. Shchipunov Y, Ivanova N, Silant'ev V. Bionanocomposites formed by in situ charged chitosan with clay. Green Chem. 2009; 11(11): 1758-61.
138. Ketelson H A, Meadows D L. Use of synthetic inorganic nanoparticles as carriers for ophthalmic and otic drugs. US Patent App. 20,050/003,014; 2002.
139. Ketelson H A, Meadows D L. Inorganic nanopartices to modify the viscosity and physical properties of ophthalmic and otic compositions. Google Patents; 2002.
140. Yang M X, Shenoy B, Disttler M, Patel R, McGrath M, Pechenov S, et al. Crystalline monoclonal antibodies for subcutaneous delivery. Proceedings of the National Academy of Sciences of the United States of America. 2003; 100(12):6934.
141. Shire S J, Shahrokh Z, Liu J. Challenges in the development of high protein concentration formulations. Journal of pharmaceutical sciences. 2004; 93(6): 1390-402.
142. Salinas B A, Sathish H A, Bishop S M, Ham N, Carpenter J F, Randolph T W. Understanding and modulating opalescence and viscosity in a monoclonal antibody formulation. Journal of pharmaceutical sciences. 2010; 99(1):82-93.
143. Nishi H, Miyajima M, Nakagami H, Noda M, Uchiyama S, Fukui K. Phase Separation of an IgG1 Antibody Solution under a Low Ionic Strength Condition. Pharmaceutical research. 2010; 27(7):1348-60.
144. Cooper C, Dubin P, Kayitmazer A, Turksen S. Polyelectrolyte-protein complexes. Current Opinion in Colloid & Interface Science. 2005; 10(1-2):52-78.

What is claimed is:

1. A method comprising:
providing a hyaluronic acid polymer, wherein the hyaluronic acid polymer has a molecular weight in the range of from about 5 kDa to about 100 kDa;
crosslinking the hyaluronic acid polymer with adipic acid dihydrazide crosslinker in a mixture comprising an 1-Ethyl-3-[3-dimethylaminopropyl]carbodiimide hydrochloride activator, water, and acetone, wherein molar reactive site ratio is greater than about 1:1;
isolating hyaluronic acid particles from the mixture, wherein at least a portion of the hyaluronic acid particles comprise a plurality of free polymer chains extending from a surface of the particle.

2. The method of claim 1 further comprising drying the hyaluronic acid particles.

3. The method of claim 1 wherein the hyaluronic acid particles are hyaluronic acid nanoparticles.

4. A hyaluronic acid particle made by the method according to claim 1.

5. An hyaluronic acid particle comprising a plurality of free polymer chains extending from a surface of the particle such that the polymer chains are capable of association with polymers or with polymer chains on a surface of other particles, wherein the hyaluronic acid particle comprises a low molecular weight hyaluronic acid polymer in the range of from about 5 kDa to about 100 kDa.

6. The particle of claim 5, wherein the particle is dried.

7. The particle of claim 5, wherein the particle is a nanoparticle.

8. A method of forming a colloidal gel without using a crosslinking agent comprising:
providing a plurality of hyaluronic acid particles, wherein at least one of the hyaluronic acid particles comprises a plurality of free polymer chains extending from a surface of the particle such that the polymer chains are capable of association with polymers or with polymer chains on a surface of other particles;
adding water to the plurality of hyaluronic acid particles; and
allowing the hyaluronic acid particles to associate.

9. The method of claim 8 wherein the plurality of hyaluronic acid particles is formulated as a dry powder.

10. The method of claim 8 wherein the plurality of hyaluronic acid particles comprises hyaluronic acid nanoparticles.

11. The method of claim 8 wherein the plurality of hyaluronic acid particles are present in solution in the range of from about 15% to about 45% w/v.

12. A colloidal gel formed by the method of claim 8.

13. A method of modifying viscosity of a polymer solution comprising: providing a polymer solution; and adding a plurality of hyaluronic acid particles to the polymer solution.

14. The method of claim 13 wherein the polymer is hyaluronic acid.

15. The method of claim 13 wherein adding the plurality of hyaluronic acid particles increases the viscosity of the polymer solution.

16. The method of claim 13 wherein adding the plurality of hyaluronic acid particles decreases the viscosity of the polymer solution.

17. The method of claim 13, wherein at least one of the hyaluronic acid particles comprises a plurality of free polymer chains extending from a surface of the particle such that the polymer chains are capable of association with polymers or with polymer chains on a surface of other particles.

18. The method of claim 13 wherein the polymer solution is an hyaluronic acid viscosupplement.

19. The method of claim 13 wherein the hyaluronic acid particles comprise hyaluronic acid nanoparticles.

20. The method of claim 13, wherein the concentration of hyaluronic acid in the polymer solution increases without a corresponding increase in the viscosity of the polymer solution.
